(19) United States
(12) Patent Application Publication
(10) Pub. No.: US 2023/0304998 A1
(43) Pub. Date: Sep. 28, 2023

(54) CELLULAR MATERIAL DETECTION PROBES

(71) Applicant: FLUORETIQ LIMITED, Bristol (GB)

(72) Inventors: Neciah DORH, Newport (GB); Josephine Ndoa DORH, Newport (GB); David BENITO-ALIFONSO, Bath (GB); Maria Carmen GALAN, Bristol (GB); James SPENCER, Bristol (GB); Jean-Baptiste VENDEVILLE, Chepstow (GB); Mathew John KYRIAKIDES, Corsham Wiltshire (GB); Henry Ralph Gordon NEWMAN, Cotham Bristol (GB); Maisie Emma HOLBROW-WILSHAW, Andoversford (GB)

(73) Assignee: FLUORETIQ LIMITED, Bristol (GB)

(21) Appl. No.: 17/923,262

(22) PCT Filed: May 7, 2021
(86) PCT No.: PCT/GB2021/051110

§ 371 Date: Nov. 4, 2022

(30) Foreign Application Priority Data
May 7, 2020 (GB) 2006791.4
Jun. 10, 2020 (GB) 2008812.6

(51) Int. Cl. G01N 33/53; G01N 33/543; G01N 33/569

(52) U.S. Cl. CPC G01N 33/5308; G01N 33/54353; G01N 33/56911; G01N 2440/38

(57) ABSTRACT

There is provided compositions for the detection of target cellular material, the composition comprising: a chemically modified substrate; at least one binding moiety, wherein the binding moiety comprises a glycan; and at least one linker covalently linked to the substrate and the binding moiety by a first and second bond. There is also provided methods of preparation of such compositions and methods for detection of target cellular material, such as for detection of pathogenic microorganisms.

### CROSS-REFERENCE TO RELATED APPLICATIONS

This application is a 35 U.S.C. § 371 National Phase Entry Application of International Application No. PCT/GB2021/051110 filed May 7, 2021, which designates the U.S. and claims benefit under 35 U.S.C. § 119(a) of G.B. Provisional Application Nos. 2006791.4 filed May 7, 2020 and 2008812.6 filed Jun. 10, 2020, the contents of which are incorporated herein by reference in their entireties.

### TECHNICAL FIELD

The present disclosure relates to cellular detection probes and particularly, but not exclusively, to detection probes for microorganisms. Aspects of the invention relate to compositions and methods for detection of cellular material.

### BACKGROUND

Infectious disease caused by bacterial and viral pathogens continue to represent a significant challenge to human and animal health. It is well-known that the emergence and growth of antimicrobial resistance (AMR) is a significant healthcare problem. As microbes continue to acquire resistance to multiple antibiotics, we face the risk of the emergence and spread of pathogenic microbes that are resistant to all known antibiotic therapies, and a return to the challenges faced in the pre-antibiotic era. Antimicrobial resistance is driven, at least in part, by inappropriate subscription of antimicrobials in both human and animal healthcare. This includes, for instance, subscription of antibiotics where there is no pathogenic bacterial infection, or subscription of broad-spectrum antibiotics when narrow-spectrum antibiotics would be suitable.

Methods for identification of the specific bacteria underlying an infection are of key importance in developing approaches to making appropriate antibiotic prescriptions that help prevent the growth of antimicrobial resistance. Reliable detection of the presence or absence of bacteria, and identification of specific species/strains, in patient samples will reduce inappropriate prescribing in primary and secondary care, respectively. Similarly, the ability to target antibiotics to specific pathogens will reduce inappropriate use of broad-spectrum antibiotics that drives emergence of both antibiotic resistance and healthcare-associated infections (HCAIs).

For example, bacterial infection largely caused by E. coli is the main cause of urinary tract infections (UTIs), generating 150 million new cases every year. The world health organisation estimates that one in two women will experience a UTI at some point in their lifetime. As common as they are, UTIs are also very poorly managed which stems from a workflow that starts treatment before actionable diagnostics are available. The impact is that 55% of patients over 65 will require a follow up visit or become hospitalised due to their infection.

The age group over 65 years accounts for 92% of the economic cost of UTIs in the UK and this key demographic is set to grow by 50% in the next 30 years (Office for National Statistics: Living Longer report 2018; HE model from NHS Digital Data 2018). Most crucially in 2018, Public Health England, advised against the use of the traditional dipstick test on the patients aged over 65 leaving few options for detection of UTI in this patient group (PHE Diagnosis of UTI: GP Quick reference guide 2018).

Current diagnostic tools used in the clinic do not meet the demands for the fast diagnostic tools needed to effectively diagnose UTI and to avoid the over prescription of unnecessary or inefficient antibiotics.

Some of the current methods to detect microorganisms in biological samples use culture technology, rapid DNA sequencing or antibody-based probes. For example WO2004/063707 describes use of antibody coated microspheres for the detection of bacteria. However, although antibody based probes are very specific, minor mutations of the antigen can affect the recognition, therefore creating non reliable probes. In addition, the quality of the antibodies can be affected by batch to batch variation especially for polyclonal antibodies. Lastly, most antibodies are not stable at room temperature or high moisture environment and, thus, kits or compositions that contain them require specific storage conditions that typically rely on consistent and reliable refrigeration (so-called cold chain integrity). Therefore, there is a need for a rapid antibody free and culture free approach to detect, for example, a potential UTI within the relatively short patient consultation window.

It is known that one class of uropathogenic bacteria use type 1 fimbriae to recognise and adhere to the host urinary tract. Type 1 fimbriae (sometimes referred to as type 1 pili) comprise a FimH adhesin protein, which recognises and binds to mannose residues on host cells [Choudhury, D. et al. Science 1999, 285, 1061-1066]. Attempts have been made to inhibit adhesion of bacteria to host cells using mannose-based antiadhesives. For instance, Sattin et al. (Trends in Biotechnology, 2016, 34:6, p 483-495) discloses use of mannose coated on nanodiamond particles as antiadhesive therapies and Yan et al. (Biomacromolecules 2015, 16, 6, 1827-1836) discloses dendrimers having high-numbers of mannose residues on each dendrimer arm as antiadhesive therapies. However, this interaction has not been used to develop a rapid detection probe for microorganisms including uropathogenic bacteria such as E. Coli. Similarly, US2007/281865 A1 and WO2005/088310 A2 describe immobilised glycan libraries on surfaces as arrays for high throughput library screening.

The present invention has been devised to mitigate or overcome at least some of the above-mentioned problems in the art.

### SUMMARY OF THE INVENTION

According to an aspect of the present invention there is provided a composition for the detection of target cellular material, the composition comprising:
a chemically modified substrate;
at least one binding moiety, wherein the binding moiety comprises a glycan; and
at least one linker covalently linked to the substrate and the binding moiety by a first and second bond.

In embodiments, the first bond is a peptide bond or a glycosidic bond.

In a further embodiment the second bond is a peptide bond or a glycosidic bond.

In embodiments the composition comprises a plurality of binding moieties.

In embodiments the composition comprises a plurality of linkers and/or spacers.

In an embodiment the second bond is a glycosidic bond. The second bond may be created via a 1,3-Huisgen-cycloaddition and may comprise a triazole.

In another embodiment the substrate is a particle or a bead; and may be selected from the group consisting of: microbeads, latex beads, magnetic beads, multi-well plates, carbon dots, gold nanoparticles, and glass surfaces.

In embodiments the substrate is functionalised with a carboxylic acid or an amine.

In another embodiment the glycan is selected from the group consisting of: Xylose; GalNAc, Gal-α-(1-4)-Gal; Gal; Man; Glc; GlcNAc; GalNAc-β-(1-4)-Gal; β-Gal; Gal-β-(1-4)-GlcNAc; GlcNAc-β-(1-3)-Gal-β-(1-4)-Glc; Glc; Gal-β-(1-3)-GalNAc; Gal-α-(1-3)-Gal; Gal-α-(1-6)-Glc; GlcNAc-3-(1-6)-GlcNAc; Fuc; GalNAc; Lac; Sorb or combinations thereof.

In another embodiment the glycan is a glycosylamine or a sugar acid.

In embodiments the linker is attached to the substrate by peptidic coupling; or via triazole formation through a 1,3-Huisgen cycloaddition reaction.

In a further embodiment the linker is a bifunctional amine, bifunctional carboxylic acid, alkanolamine, alkanolamide, or aminocarboxylic acid.

In another embodiment the chemically modified substrate comprises a plurality of glycans and linkers, optionally wherein the plurality of glycans are identical.

In another embodiment the linker is selected from the group consisting of: 2-azidoethanol; 3-azidopropanol; 5-azidopentanol; 7-azidoheptanol; 10-azidodecanol; 2-aminoethanol; 3-aminopropanol; 5-aminopentanol; 7-aminoheptanol; 10-aminodecanol; 4,7,10-trioxa-1,13-diaminotridecane and carboxylated variants thereof and the following structures or combinations thereof:

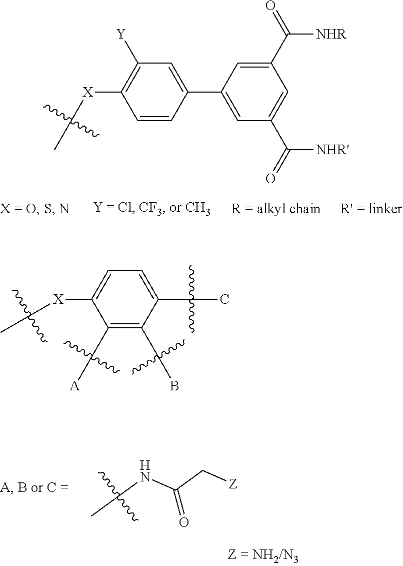

The linker is for example of the following structure:

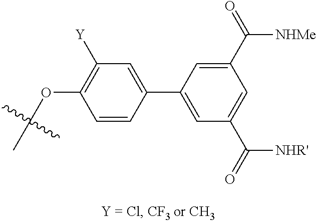

In embodiments the composition comprises a spacer.

In a further embodiment the spacer is selected from the group consisting of: 2-(2-(2-(2-aminoethoxy)ethoxy)ethoxy)ethanol or 4,7,10-trioxa-1,13-diaminotridecane or combinations thereof.

In another embodiment the target cellular material is the cell surface of a microorganism, typically a bacteria or a yeast, suitably a pathogenic bacteria.

In embodiments the microorganism is selected from the group consisting of: Escherichia coli, Klebsiella pneumoniae, Proteus mirabilis, Enterobacter cloacae, Proteus vulgaris, Klebsiella aerogenes, Citrobacter freundii, Citrobacter kosieri, Staphylococcus saprophyticus, Staphylococcus aureus, Staphylococcus epidermidis, Enterococcus faecalis, Streptococcus agalactiae, Streptococcus pyogenes, Candida albicans, Neisseria gonorrhoeae and Treponema. In a second aspect there is provided a method for the detection of target cellular material, the method comprising:
(i) providing a sample comprising cellular material;
(ii) adding to the sample a composition comprising a chemically modified substrate, a binding moiety comprising a glycan, and
   a linker covalently linked to the substrate and the binding moiety by a first and second bond;
   wherein selective binding of the target cellular material to the glycan causes agglutination; and
(iii) measuring the level of agglutination in order to determine the presence of target cellular material.

In embodiments step (iii) further comprises determining the concentration of the target cellular material.

In embodiments the substrate comprises a particle or a bead.

In a further embodiment the substrate is selected from the group consisting of: microbeads, latex beads, magnetic beads, and multi-well plates and glass substrates.

In another embodiment the glycan is selected from the group consisting of: Xylose; GalNAc, Gal-α-(1-4)-Gal; Gal; Man; Glc; GlcNAc; GalNAc-β-(1-4)-Gal; β-Gal; Gal-β-(1-4)-GlcNAc; GlcNAc-β-(1-3)-Gal-β-(1-4)-Glc; Glc; Gal-β-(1-3)-GalNAc; Gal-α-(1-3)-Gal; Gal-α-(1-6)-Glc; GlcNAc-3-(1-6)-GlcNAc; Fuc; GalNAc; Lac; Sorb or combinations thereof.

In embodiments the linker is attached to the substrate by peptidic coupling; or via triazole formation through a 1,3-Huisgen cycloaddition reaction.

In a further embodiment the target cellular material is the cell surface of a microorganism, suitably a bacteria or yeast, typically a bacteria pathogenic.

In another embodiment lectin protein on the cell surface of the microorganism binds to the glycan.

In embodiments the microorganism is selected from the group consisting of: Escherichia coli, Klebsiella pneumoniae, Proteus mirabilis, Enterobacter cloacae, Proteus vulgaris, Klebsiella aerogenes, Citrobacter freundii, Citrobacter kosieri, Staphylococcus saprophyticus, Staphylococcus aureus, Staphylococcus epidermidis, Enterococcus faecalis, Streptococcus agalactiae, Streptococcus pyogenes, Candida albicans, Neisseria gonorrhoeae and Treponema and combinations thereof.

In a third aspect there is provided a method for the preparation of a composition for the detection of target cellular material, the method comprising:
providing a chemically modified substrate;
covalently attaching a linker to the chemically modified substrate by a first bond; and covalently attaching a binding moiety comprising a glycan to the substrate by a second bond.

In embodiments, the first bond is a peptide bond or a glycosidic bond.

In a further embodiment the second bond is a peptide bond or a glycosidic bond.

In embodiments the composition comprises a plurality of binding moieties.

In embodiments the composition comprises a plurality of linkers and/or spacers.

In an embodiment the second bond is a glycosidic bond. The second bond may be created via a 1,3-Huisgen-cycloaddition and may comprise a triazole.

In another embodiment the substrate is a particle pr a bead and may be selected from the group consisting of: microbeads, latex beads, magnetic beads, multi-well plates, carbon dots, gold nanoparticles, and glass surfaces.

In embodiments the substrate is carboxylic acid or amine functionalised.

In another embodiment the glycan is selected from the group consisting of: Xylose; GalNAc, Gal-α-(1-4)-Gal; Gal; Man; Glc; GlcNAc; GalNAc-β-(1-4)-Gal; 3-Gal; Gal-β-(1-4)-GlcNAc; GlcNAc-β-(1-3)-Gal-β-(1-4)-Glc; Glc; Gal-β-(1-3)-GalNAc; Gal-α-(1-3)-Gal; Gal-α-(1-6)-Glc; GlcNAc-3-(1-6)-GlcNAc; Fuc; GalNAc; Lac; Sorb or combinations thereof.

In another embodiment the glycan is a glycosylamine or a sugar acid.

In embodiments the linker is attached to the substrate by peptidic coupling or triazole formation through a 1,3-Huisgen cycloaddition.

In a further embodiment the linker is a bifunctional amine, bifunctional carboxylic acid, alkanolamine, alkanolamide, or aminocarboxylic acid.

In another embodiment the chemically modified substrate comprises a plurality of glycans and linkers, optionally wherein the plurality of glycans are identical.

In another embodiment the linker is selected from the group consisting of: 2-azidoethanol; 3-azidopropanol; 5-azidopentanol; 7-azidoheptanol; 10-azidodecanol; 2-aminoethanol; 3-aminopropanol; 5-aminopentanol; 7-aminoheptanol; 10-aminodecanol; 4,7,10-trioxa-1,13-diaminotridecane and carboxylated variants thereof and the following structures or combinations thereof:

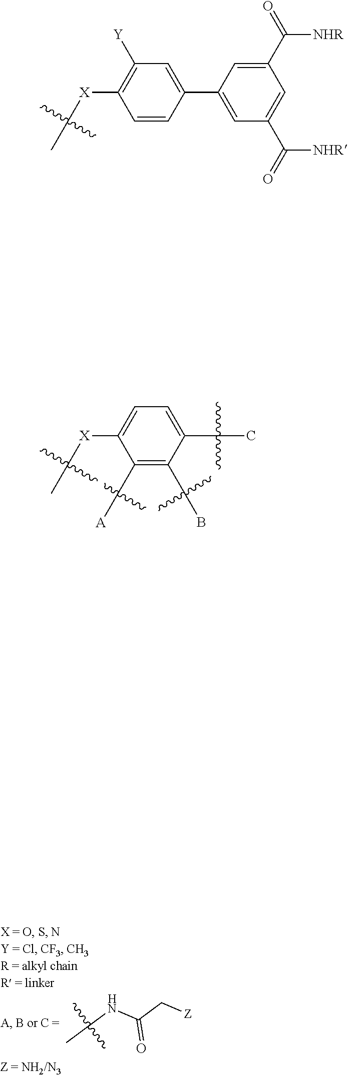

The linker may be for example of the following structure:

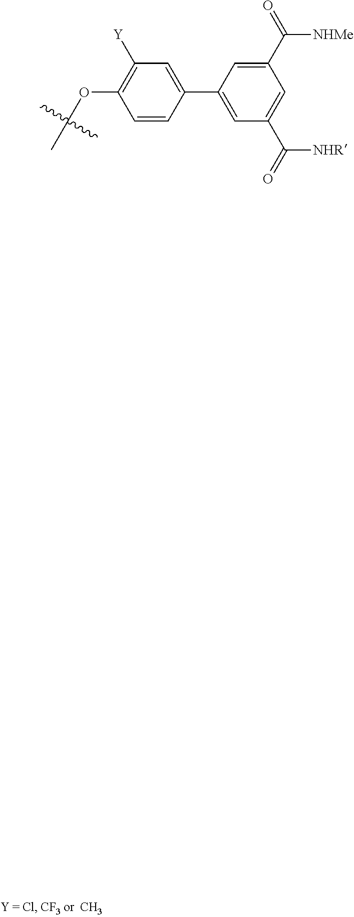

In embodiments the composition comprises a spacer.

In a further embodiment the spacer is selected from the group consisting of: 2-(2-(2-(2-aminoethoxy)ethoxy)ethoxy)ethanol or 4,7,10-trioxa-1,13-diaminotridecane or combinations thereof.

In another embodiment the target cellular material is the cell surface of a microorganism, typically pathogenic bacteria.

In embodiments the microorganism is selected from the group consisting of: Escherichia coli, Klebsiella pneumoniae, Proteus mirabilis, Enterobacter cloacae, Proteus vulgaris, Klebsiella aerogenes, Citrobacter freundii, Citrobacter kosieri, Staphylococcus saprophyticus, Staphylococcus aureus, Staphylococcus epidermidis, Enterococcus faecalis, Streptococcus agalactiae, Streptococcus pyogenes, Candida albicans, Neisseria gonorrhoeae and Treponema.

In a fourth aspect there is provided a kit for the detection of cellular material comprising:
(i) a composition comprising a chemically modified substrate, a binding moiety comprising a glycan, and a linker covalently linked to the substrate and the binding moiety,
(ii) a buffer solution; and
(iii) a solvent.

In a further aspect there is provided composition for the detection of target cellular material, the composition comprising:
a chemically modified substrate;
at least one binding moiety, wherein the binding moiety comprises a glycan; and
wherein the chemically modified substrate is covalently linked to the binding moiety by a first bond.

In further aspects of the invention, there is also provided a probe for detecting bacteria having type 1 fimbriae, wherein the probe comprises a substrate to which is bonded a molecule of Formula III:

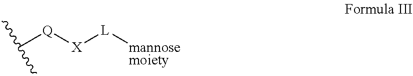

wherein:

Q is a bond or a linker;

X is other than hydrocarbylene and is of 1 atom in length;

L is a hydrocarbylene group of 1 to 10 atoms in length; and

the mannose moiety comprises at least one mannose residue.

In embodiments L is of formula (CR₂)ₙ, wherein n is 1 to 10 and each R is independently selected from:
—H or C₁₋₆ alkyl, alkenyl, alkynyl, cycloalkyl or aryl; and/or
R₂ of a CR₂ is together an alkene of a C₁₋₆ alkyl, alkenyl, alkynyl, cycloalkyl or aryl; and/or consecutive CR₂ groups define an alkynyl group or alkenyl group, or part of a cycloalkyl or aryl group.

In embodiments each R is —H.

In embodiments n is 3 to 9, suitably 4 to 8, more suitably 7.

In embodiments X is selected from —C(O)—, —O—, —NR¹—, —S—, —S(O)—, —S(O)(O)—, or —P(O)OR¹— wherein each R¹ is independently selected from H or C₁₋₆ alkyl.

In embodiments Q comprises the formula —C(O)—Y—C(O)—, wherein Y is selected from an optionally substituted alkyl, optionally substituted cycloalkyl, optionally substituted alkenyl, optionally substituted alkynyl, optionally substituted aryl, optionally substituted heteroaryl and optionally substituted heterocyclyl.

In embodiments Y is (—Z—)ₙ, wherein each —Z— is independently chosen from —CR²₂—, —O—, or —NR²—, wherein each R² is independently selected from —H, —OH, —NH₂ or —C₁₋₆ alkyl, and n is from 1 to 30.

In embodiments the mannose moiety is selected from monomannose or a polysaccharide comprising at least one mannose residue.

In embodiments the mannose moiety is connected to L by a glycosidic heteroatom.

In embodiments the substrate is a particle.

In embodiments the particle is fluorescent.

In embodiments, the particle has an average diameter of 0.1 nm to 100 μm.

In embodiments the particle is a fluorescent dot, suitably a fluorescent carbon dot.

In embodiments wherein the fluorescent dot has an average diameter of 0.1 nm to 10 nm, suitably 0.5 nm to 5 nm, more suitably 2 nm to 3 nm.

In embodiments the particle is a bead comprising a plastics material, suitably wherein the plastics material is latex.

In embodiments the bead has an average diameter of 0.1 μm to 100 μm, suitably 1 μm to 50 μm, more suitably about 10 μm.

In embodiments molecules of Formula III are bound to 50 to 100% of the substrate surface.

There is further provided a composition comprising a probe according to any preceding aspect or embodiment and a buffer, suitably wherein the composition is in solution.

In addition, there is provided for the use of a probe or composition in accordance with the preceding embodiments for the detection of bacteria having type 1 fimbriae.

There is also provided for a method of detecting bacteria having type 1 fimbriae in a test sample, the method comprising the steps of:
a) providing a probe according to any one of claims 1 to 17 or a composition according to claim 18;
b) contacting the probe or composition with a test sample;
c) providing sufficient time for the probe to bind to the bacteria; and
d) detecting the presence of the probe-bacteria complex.

In embodiments the test sample is an isolated body fluid, isolated tissue sample, foodstuff, or surface swab, suitably wherein the test sample is a urine sample.

In embodiments wherein the substrate is a particle and step d) comprises the detection of clusters of probe-bacteria complex.

In embodiments the substrate is fluorescent particle and step d) comprises sequentially exciting the substrate using at least two different peak emission wavelengths, and detecting the fluorescence signal.

Finally there is provided a kit comprising a probe according to any one of the preceding aspects, embodiments and compositions, and an apparatus for contacting the probe or composition with a test sample.

In embodiments the kit further comprises a detector for detection of the presence of the probe-bacteria complex.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

One or more embodiments of the invention will now be described, by way of example only, with reference to the accompanying drawings, in which:

FIG. 1 is a scheme describing the nature of a linker according to an embodiment of the invention.

FIG. 2 is a scheme describing the functionalisation of a chemically modified substrate.

FIG. 3 is a scheme describing the functionalisation of a chemically modified substrate via the Huisgen cycloaddition reaction.

FIG. 4 is a general scheme describing the functionalisation of a glycan by glycosylation reactions.

FIG. 5 is a scheme describing the functionalisation of C7 mannose in order to attach a glycan to the chemically modified substrate.

FIG. 6 is a scheme describing the functionalisation of C-3-GalNAc in order to attach the glycan to the chemically modified substrate.

FIG. 7 shows microscope images of samples according to one embodiment of the invention that contains micro bead probes only (FIGS. 7A and 7B) and samples containing bacteria bound to micro bead probe (FIGS. 7C and 7D). More specifically, FIGS. 7A and B show agglutination of a tryptic soy broth blank with NH₂-Xylose conjugated 10 μl latex beads at 4× (A) and 10× (B) magnification; and FIGS. 7C and D show agglutination of P. mirabilis NCTC 11938 with NH₂-Xylose conjugated 10 μl latex beads at 4× (C) and 10× (D) magnification.

FIG. 8 shows microscope images of samples containing different microorganisms agglutinated by the 1 NH₂-Xyl probe (FIGS. 8A to 8C). More specifically, FIG. 8A shows the use of the 1 NH₂-Xyl probe in the presence of P. mirabilis; FIG. 8B shows the use of the same probe in the presence of E. coli and FIG. 8C shows the use of the same probe in the presence of K. pneumoniae.

FIG. 9 is a schematic showing example methods of synthesising probes in accordance with embodiments of the invention.

FIG. 10 is a schematic showing an example method of synthesising a fluorescent carbon dot according to embodiments of the invention.

FIG. 11 shows microscope images of test samples mixed with a microsphere probe according to the invention. The test sample in FIG. 11A is without bacteria and FIG. 11B shows a test sample with bacteria having type 1 fimbriae. In both FIGS. 11A and 11B the left image is the original bright-field image and the right image is after processing.

FIG. 12 shows the result of an analysis of number of clusters against varying alky chain lengths of an alkyl group L according to Formula III.

FIG. 13 shows the results of studies where Saccharomyces cerevisiae yeast is used as a mannose-displaying cell to agglutinate BW25113 E. coli (i.e. E. coli having type 1 fimbriae). The results of a FimH knockout BW25113 E. coli is also shown. Agglutination was assessed for the following column headings: Control denotes no CDs added; G-CDs, unfunctionalised CDs; L-B-CDs, lactosylated blue CDs; M-B-CDs, mannosylated CDs.

FIG. 14 shows confocal images of quantum dot probes incubated with the BW25113 E. coli and the FimH knockout for 1 hour before fixation. FIG. 6A shows the fluorescence channel showing labelling of the E. coli. FIGS. 6B and 6C show an overlay of the fluorescence and bright field channels of E. coli (B) and fimH knockout E. coli (C).

FIG. 15 is a line graph and shows dilution series or concentration gradient of: E. coli conjugated to the C7-mannose probe using EDC peptide coupling (A) and E. coli conjugated to the C7-mannose probe using CuACC click chemistry.

FIG. 16 is a line graph and shows a dilution series or concentration gradient of E. coli conjugated to the C7-mannose probe (A); K. pneumoniae conjugated to the C7-mannose probe (B): P aeruginosa conjugated to the C3-Fucose probe (C) and P. mirabilis conjugated to the C3-GlcNAc probe (D).

FIG. 17 is a bar graph and shows total cluster sizes (TCS) as a measure for specificity of various probes for E. coli (A); K. pneumoniae (B); P aeruginosa (C); and P. mirabilis (D).

FIG. 18 is a bar graph and shows total cluster sizes (TCS) as a measure for specificity of various strains of the same species: E. coli (A); P. mirabilis (B); and K. pneumoniae (C).

FIG. 19 describes the installation of an aromatic linker on a galactose unit prior to conjugation to a microsphere or other solid support. The procedure is generally described by Titz et al; Organic biomolecular chemistry, 2016, 14, 7933-7948.

### DETAILED DESCRIPTION

There is presented compositions and methods for the detection of cellular material for example microorganisms.

Prior to setting forth the compositions and methods, a number of definitions are provided that will assist in the understanding of the invention.

### Definitions

As used herein, the term “comprising” means any of the recited elements are necessarily included and other elements may optionally be included as well. “Consisting essentially of” means any recited elements are necessarily included, elements that would materially affect the basic and novel characteristics of the listed elements are excluded, and other elements may optionally be included. “Consisting of” means that all elements other than those listed are excluded. Embodiments defined by each of these terms are within the scope of this invention.

As used herein, the term “substantially” refers to the complete or nearly complete extent or degree of an action, characteristic, property, state, structure, item, or result. For example, an object that is “substantially” coplanar with another object would mean that the object is either completely coplanar or nearly completely coplanar, perhaps varying by a few degrees of variation from complete conformity. The exact allowable degree of deviation from absolute completeness may in some cases depend on the specific context, as would be understood to the person of skill in the art. However, in general terms the nearness of conformity to the absolute will be such as to have the same overall result—e.g. functional equivalence—as if total conformity were achieved.

The term “hydrogen” or “hydrogen atom” as used herein refers to a —H moiety.

The term “alkyl” as used herein refers to a hydrocarbon compound having from 1 to 12 carbon atoms (unless otherwise specified), which may be aliphatic or alicyclic, which may be saturated or unsaturated (e.g. partially unsaturated or fully unsaturated), and which may be linear or branched. Thus, the term “alkyl” includes the sub-classes alkenyl, alkynyl, cycloalkyl, cycloalkenyl and cylcoalkynyl below.

In the context of alkyl groups, the prefix C₁₋₁₂ denotes the number of carbon atoms, or range of number of carbon atoms present in that group. Thus, the term “C₁₋₁₂ alkyl” refers to an alkyl group having from 1 to 12 carbon atoms. The first prefix may vary according to the nature of the alkyl group. Thus, if the alkyl group is an alkenyl or alkynyl group, then the first prefix must be at least 2 (e.g. C₂₋₁₂). For cyclic (e.g. cycloalkyl, cycloalkenyl, cylcoalkynyl) or branched alkyl groups, the first prefix must be at least 3 (e.g. C₃₋₁₂).

Examples of saturated alkyl groups include methyl (C₁), ethyl (C₂), propyl (C₃), butyl (C₄), pentyl (C₅), hexyl (C₆), heptyl (C₇), octyl (C₈), nonyl (C₉) and decyl (C₁₀). Examples of saturated linear alkyl groups include, but are not limited to, methyl (C₁), ethyl (C₂), n-propyl (C₃), n-butyl (C₄), n-pentyl (amyl) (C₅), n-hexyl (C₆), and n-heptyl (C₇). Examples of saturated branched alkyl groups include iso-propyl (C₃), iso-butyl (C₄), sec-butyl (C₄), tert-butyl (C₄), iso-pentyl (C₅), and neo-pentyl (C₅).

The term “alkenyl” refers to an alkyl group having one or more carbon-carbon double bonds.

Examples of unsaturated alkenyl groups include ethenyl (vinyl, —CH═CH₂), 1-propenyl (—CH═CH—CH₃) and 2-propenyl (allyl, —CH—CH═CH₂).

The term “alkynyl” refers to an alkyl group having one or more carbon-carbon triple bonds. Examples of unsaturated alkynyl groups include, but are not limited to, ethynyl (ethinyl, —C═CH) and 2-propynyl (propargyl, —CH₂—C≡CH).

The term “cycloalkyl” refers an alkyl group which is also a cyclyl group; that is, a monovalent moiety obtained by removing a hydrogen atom from an alicyclic ring atom of a carbocyclic compound (i.e. a compound where all of the ring atoms are carbon atoms). The ring may be saturated or unsaturated (e.g. partially unsaturated or fully unsaturated), which moiety has from 3 to 12 carbon atoms (unless otherwise specified). Thus, the term “cycloalkyl” includes the sub-classes cycloalkenyl and cycloalkynyl. In an embodiment, each ring has from 3 to 7 ring carbon atoms. Examples of cycloalkyl groups include those derived from (i) saturated monocyclic hydrocarbon compounds: cyclopropane (C3), cyclobutane (C4), cyclopentane (C5), cyclohexane (C6), cycloheptane (C7) and methylcyclopropane (C4); (ii) unsaturated monocyclic hydrocarbon compounds: cyclopropene (C3), cyclobutene (C4), cyclopentene (C5), cyclohexene (C6), methylcyclopropene (C4) and dimethylcyclopropene (C5); (iii) saturated polycyclic hydrocarbon compounds: thujane (C10), carane (C10), pinane (C10), bornane (C10), norcarane (C7), norpinane (C7), norbornane (C7), adamantane (C10), decalin (C10); (iv) unsaturated polycyclic hydrocarbon compounds: camphene (C10), limonene (C10), pinene (C10); and (v) polycyclic hydrocarbon compounds having an aromatic ring: indene (C9), indane (C9) and tetraline (C10).

In an embodiment, a reference to an alkyl group described herein is a C₁₋₁₂ alkyl group, such as a C₁₋₈ alkyl group, for example a C₁₋₆ alkyl group, or a C₁₋₄ alkyl group. The alkyl groups in the invention can be saturated alkyl groups or saturated cycloalkyl groups, for example saturated, unbranched alkyl groups.

The phrase “optionally substituted” as used herein refers to a parent group which may be unsubstituted or which may be substituted with one or more, for example one or two, substituents. The substituents on an “optionally substituted” group may for example be selected from alkyl, cycloalkyl, alkenyl, alkynyl, aryl, heteroaryl and heterocyclyl groups; carboxylic acids and carboxylate ions; carboxylate esters; carbamates; alkoxyl groups; ketone and aldehyde groups; amine and amide groups; —OH; —CN; —NO₂; and halogens.

The term “substituents” is used herein in the conventional sense and refers to a chemical moiety, which is covalently attached to, or if appropriate, fused to, a parent group.

In some embodiments, substituents can themselves be substituted. For example, a C1-12 alkyl group may be substituted with, for example, hydroxy (referred to as a hydroxy-C1-12 alkyl group) or a halogen atom (referred to as a halo-C1-12 alkyl group), and a C1-12 alkoxy group may be substituted with, for example, a halogen atom (referred to as a halo-C1-12 alkoxy group).

The term “aryl” as used herein refers to an aromatic ring atom of an aromatic compound, which moiety has from 6 to 10 ring carbon atoms (unless otherwise specified). In an embodiment, the aryl group is a phenyl group.

The term “heteroaryl” as used herein refers to a heteroaromatic compound, which moiety may for example be a monocyclic or bicyclic group. The heteroaryl moiety may contain from 1 to 12 carbon atoms (unless otherwise specified) and one or more N, O or S atoms. The heteroaryl moiety may be a 5 or 6-membered ring containing one or more N atoms.

The term “heterocyclyl” as used herein refers to a monovalent moiety obtained by removing a hydrogen atom from a ring atom of a heterocyclic compound, which moiety may for example be a monocyclic or bicyclic group. The heterocyclyl group may contain from 1 to 12 carbon atoms (unless otherwise specified) and one or more N, O or S atoms.

The term “alkoxy” used herein refers to an alkyl-oxy group, where the alkyl group is as defined above and has from 1 to 12 carbon atoms (unless otherwise specified). In an embodiment, the alkyl moiety in an alkoxy group is a saturated alkyl group or a saturated cycloalkyl group. In an embodiment, the alkyl moiety is a saturated, unbranched alkyl group. Examples of C1-12 alkoxy groups include —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Where present, the functional groups C(O)O and C(O)NR can be found in either orientation. In other words, C(O)O represents —C(O)O— and —OC(O)—; and C(O)NR represents —C(O)NR— and —NRC(O)—.

The term “target cellular material” as used herein refers to any cellular material, including whole cells, or extracts of whole cells. The target cellular material may comprise the cell wall, cell membrane or capsular membrane of a microorganism, typically a pathogen. The surface of the cellular material may comprise cell-surface components that facilitate adhesion to other cells. Such cell surface components may be lectin proteins capable of recognising glycans, for example. The definition of target cellular material further extends to viruses—e.g. virion particles—equally capable of expressing glycan binding lectins in order to adhere to host cells.

The wording “chemically modified substrate” refers to any platform material which has been altered by way of chemical functionalisation. The chemically modified substrate may be a functionalised bead such as a carboxylated and/or aminated microbead (also referred to microsphere). The microbead may be made of polymeric material such as latex but can be made from any other material including glass, carbon nanotubes, metals and metal alloys. The chemically modified substrate may be any surface which has been chemically functionalised such as the bottom surface of a well plate or a surface within a microfluidic chip.

The term “binding moiety” as used herein refers to any chemical moiety capable of interacting with cellular material. In cases where the cellular material is the cell surface of a microorganism or virus, the binding moiety is a glycan. The glycan may be naturally occurring but the glycan may also be synthetically modified. Examples beyond naturally occurring glycans may be deoxy- and fluoro-glycan analogues.

The wording “linker” refers to an at least bifunctional (bidentate) chemical moiety (i.e. a chemical moiety having at least two functional groups) capable of ‘linking’ a substrate with a binding moiety. The linker may act as a bridging group between the substrate and the binding moiety. The design of the linker may modulate the binding moiety coverage on the substrate.

The term “spacer” as used herein refers to any chemical moiety bound to the substrate which however, does not comprise a glycan binding moiety. Spacers may be installed to fine tune the glycan coverage on the substrate and improve the composition's sensitivity towards a certain target cellular material. For example, spacers and linkers may be bound to the surface of the substrate in a certain ratio beneficial for detection of a specific cellular material. The spacer may be a particularly bulky chemical moiety, allowing a defined amount of cellular material to be recognised by the glycan binding moiety. Alternatively, the spacer may be a long chain chemical moiety which again will only allow a defined amount of cellular material to be recognised by the glycan binding moiety.

The term “first bond” refers to the covalent chemical bond between the substrate and the linker. This first bond may be, but is not limited to, a peptide bond. In this context the term “second bond” refers to the covalent chemical bond between the linker and the binding moiety. This second bond may be but is not limited to a peptide bond. The first and second bonds may be of the same type or of a different type. For example the first bond may be an ester bond and the second bond may be a peptide (or amide) bond. Typically, the first and second bonds are both peptide bonds or a glycosidic bond (typically O-glycosidic) and a peptide bond. Other ways of achieving a covalent first and second bond may be reductive amination and 1,3-Huisgen triazole formation (1,3-Huisgen-cycloaddition).

The wording “covalently” describes the bond between at least two chemical moieties. A covalent bond (as opposed to an ionic bond) is a chemical bond that involves the sharing of electron pairs between atoms. For example, the substrate with the linker and the linker with the binding moiety may be connected by two covalent bonds. In certain embodiments, the glycan may not always be immobilised on the substrate by means of a covalent bond. The glycan could be immobilised by for example adsorption or ionic bond encapsulation.

The terminology “selective binding” as referred to herein refers to the binding of the binding moiety e.g. a glycan to the cellular material. For example, the cellular material may be coated in lectin proteins in order to facilitate adhesion to cells. These lectin proteins recognise different glycan entities depending on the type of cellular material. Therefore, by designing a composition with a specific glycan coating in form of the binding moiety, the composition is able to act as a probe and recognises at least the presence of the cellular material. More specifically, the cell surface of many microorganisms is coated in proteins which recognise a variety of different glycan entities such as the lectin FimH present on the cell surface of E. coli which recognises D-mannose.

The term “agglutination” generally describes clumping of particles to form an agglomerated complex. In the context of this application agglutination occurs once the target cellular material is reversibly bound to the composition which may be in the form of lectin-glycoconjugates. The composition itself does not clump in the absence of target cellular material.

The term “microorganism” as referred to herein denotes bacterial and fungal species (e.g. yeast) and includes, but is not limited to: Escherichia, Staphylococcus, Salmonella, Klebsiella, Enterobacter, Serratia, Citrobacter, Proteus, Coagulase-negative Staphylococcus, Pseudomonas, Bacillus, Clostridium, Streptococcus, Listeria, Acinetobacter, Klebsiella, Proteus, Salmonella, Propionibacterium, Heliobacter, Porphyromonas, Prevotella, Aggregatibacter, Saccharomyces, Candida, and Aspergillus. Specific species can include Escherichia coli, Streptococcus mutans, Streptococcus pneumoniae, Neisseria Gonorrhoeae, Meningococcus, Haemophilus influenzae, Staphylococcus aureus, methicillin-resistant Staphylococcus aureus (MRSA), Staphylococcus saprophyticus, Staphylococcus epidermidis, Pseudomonas aeruginosa, Bacillus cereus, Bacillus subtilis, Bacillus megaterium, Clostridium difficile, Streptococcus Enterococcus, Acinetobacter baumannii, Pseudomonas aeruginosa, Klebsiella pneumoniae, Proteus vulgaris, Proteus mirabilis, Salmonella, Propionibacterium acnes, Helicobacter pylor, Porphyromonas gingivalis, Prevotella intermedia, Aggregatibacter actinonycetemcomitans, Saccharomyces cerevisiae, Candida albicans, Aspergillus niger, Neisseria gonorrhoeae and Treponema pallidum. Specific strains include, but are not limited to: Escherichia coli Type 1, P, S, CFA/1, K1, and K99; Staphylococcus aureus NCTC 4135, MRSA-US-300 of CA-MRSA, IS853 of HA-MRSA, Staphylococcus epidermidis NCTC 11964, Bacillus cereus NCTC 11143, Clostridium difficile NCTC 11204, Acinetobacter baumannii NCTC 12156, Pseudomonas aeruginosa NCTC 11143, Klebsiella pneumoniae NCTC 9633 and Proteus vulgaris NCTC CN 329.

Typically, the targeted microorganisms are selected from: Escherichia coli, Klebsiella pneumoniae, Proteus mirabilis, Enterobacter cloacae, Proteus vulgaris, Klebsiella aerogenes, Citrobacter freundii, Citrobacter kosieri, Staphylococcus saprophyticus, Staphylococcus aureus, Staphylococcus epidermidis, Enterococcus faecalis, Streptococcus agalactiae, Streptococcus pyogenes, Candida albicans, Neisseria gonorrhoeae and Treponema pallidum.

Microorganisms may be pathogenic or non-pathogenic (e.g. including commensal species). They may include microorganisms comprised within a microbiome or biofilm. In addition, microorganisms may be of nosocomial origin.

The term “urinary tract infection (UTI)” as referred to herein is an infection in any part of the genito-urinary system which may include the kidneys, ureters, bladder and urethra. Most commonly urinary tract infections are located in the lower urinary tract. The infection is predominantly caused by gram-negative and gram-positive bacteria including but not limited to Escherichia coli (E. coli). In the clinic, E. coli accounts for about 80% of cases. The gram-negative genera Klebsiella, Proteus, Enterobacter, Pseudomonas, and Serratia account for about 40%, and the gram-positive bacterial cocci E. faecalis, S. saprophyticus, and Staphylococcus aureus account for the remaining species causing UTI.

Without wishing to be bound by theory it is understood that glycans play an important role in the adhesion process of pathogenic organisms to the tissue of the host. The adhesion is mediated by lectin proteins present on the surface of the infectious organism which can bind to the glycans on the surface of the host tissue (Sharon and Ofek, “Safe as mother's milk: Carbohydrates as future anti-adhesion drugs for bacterial diseases”, Glycoconjugate Journal 17, 659-664, 2000).

Species specific labelling can be achieved through attachment of specific glycans to the surface of the substrate for the detection of targeted microorganisms in a physiological sample.

Glycan Probes

The composition or probe, in specific embodiments of the invention, comprises at least one group that conforms to general Formula I, set out below:

A—[C]ₙ—X I

wherein A includes a substrate tethered to the glycan X by a linker group C of n atoms in length. The linker group is covalently connected to the substrate and the glycan by a first and second bond. Typically A comprises a chemically functionalised surface such as a carboxylated or aminated microbead. In a specific embodiment of the invention A comprises a carboxylated microbead which may be made of latex. The glycan can be selected suitably from: xylose, Gal-α-(1-4)-Gal; Gal; Man; Glc; GlcNAc; GalNAc-β-(1-4)-Gal; β-Gal; Gal-β-(1-4)-GlcNAc; GlcNAc-β-(1-3)-Gal-β-(1-4)-Glc; Glc; Gal-β-(1-3)-GalNAc; Gal-α-(1-3)-Gal; GalNac, Gal-α-(1-4)-Gal, Gal-α-(1-6)-Glc; GlcNAc-β-(1-6)-GlcNAc, Fuc, GalNAc, Lac, Sorb or combinations thereof.

The length of the linker group is such that n is suitably between about 5 and about 100 atoms; more suitably between about 6 and 80 atoms; more typically between about 7 and about 60 atoms; suitably between about 9 and about 40 atoms. The linker group is such that it has between about 1 and about 30 carbon atoms; more suitably between about 2 and 20 carbon atoms; more typically between about 2 and about 10 carbons. In some embodiments, a linker group may not always be present such that the glycan is directly tethered to the substrate in which case n of Formula I above equals zero.

Linker group C may be hydrocarbylene group of 1 to 10 atoms in length. By hydrocarbylene, we are referring to hydrocarbon groups such as alkyl, alkenyl, alkynyl and aryl groups. By length, we are referring to the atoms in the chain between the binding moiety and the substrate. Further hydrocarbylene side groups may be present. Where there are different options for following the chain length between the mannose moiety and the substrate (for example, the chain contains a cyclopentyl group), the shortest chain length should be counted. It is preferred that L has a molecular weight below 500, 400 or 300 Da, and suitably below 200 Da.

In a preferred embodiment, the linker group is of formula (CR₂)ₙ, wherein n is 1 to 10 and each R is independently selected from: H or C₁₋₆ alkyl, alkenyl, alkynyl, cycloalkyl or aryl; and/orR₂ of a CR₂ is together an alkene of a C₁₋₆ alkyl, alkenyl, alkynyl, cycloalkyl or aryl; and/or consecutive CR₂ groups define an alkynyl group or alkenyl group, or part of a cycloalkyl or aryl group.

By this, we mean that the linker group C can comprise alkyl, alkenyl, alkynyl, cycloalkyl or aryl groups, or combinations thereof, in the chain between the binding moiety and the substrate. In addition, side groups may also comprise alkyl, alkenyl, alkynyl, cycloalkyl or aryl groups, or combinations thereof. Suitably, each R is —H and/or consecutive CR₂ groups define an alkynyl group, alkenyl group or aryl group.

In a particularly preferred embodiment, each R is —H. In other words, it is preferred that the linker group is C₁₋₁₀ alkyl. Where the linker group is is of formula (CR₂)ₙ, n can be at least 1, 2, 3, 4, 5, 6, 7, 8 or 9 and can up to 10, 9, 8, 7, 6, 5, 4, 3 or 2. Furthermore, n can be: 1 to 9, 1 to 8, 1 to 7, 1 to 6, 1 to 5, 1 to 4, 1 to 3, 1 to 2, 1, 2 to 10, 2 to 9, 2 to 8, 2 to 7, 2 to 6, 2 to 5, 2 to 4, 2 to 3, 2, 3 to 10, 3 to 9, 3 to 8, 3 to 7, 3 to 6, 3 to 5, 3 to 4, 3, 4 to 10, 4 to 9, 4 to 8, 4 to 7,4 to 6, 4 to 5, 4,5 to 10, 5 to 9, 5 to 8, 5 to 7, 5 to 6, 5, 6 to 10, 6 to 9, 6 to 8, 6 to 7, 6, 7 to 10, 7 to 9, 7 to 8, 7, 8 to 10, 8 to 9, 8, 9 to 10, 9, or 10.

In a preferred embodiment, n is 3 to 9, suitably at least 4, more suitably 4 to 8. In a particularly preferred embodiment, n is 7.

According to specific embodiments of the invention, the linker group may be a bifunctional aminoalkyl compound, for example a C2, C3, C5 or C10 aminoalkyl compound. Suitably the linker group may be a bifunctional alkanolamine or alkanolamide such as 2-aminoethanol; 3-aminopropanol; 5-aminopentanol; 7-aminoheptanol; 10-aminodecanol 4,7,10-trioxa-1,13-diaminotridecane and carboxylated variants thereof or the following structure or combinations thereof:

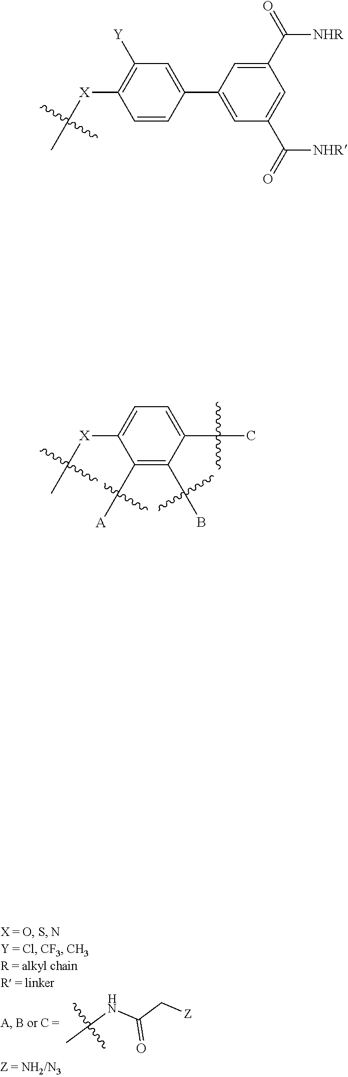

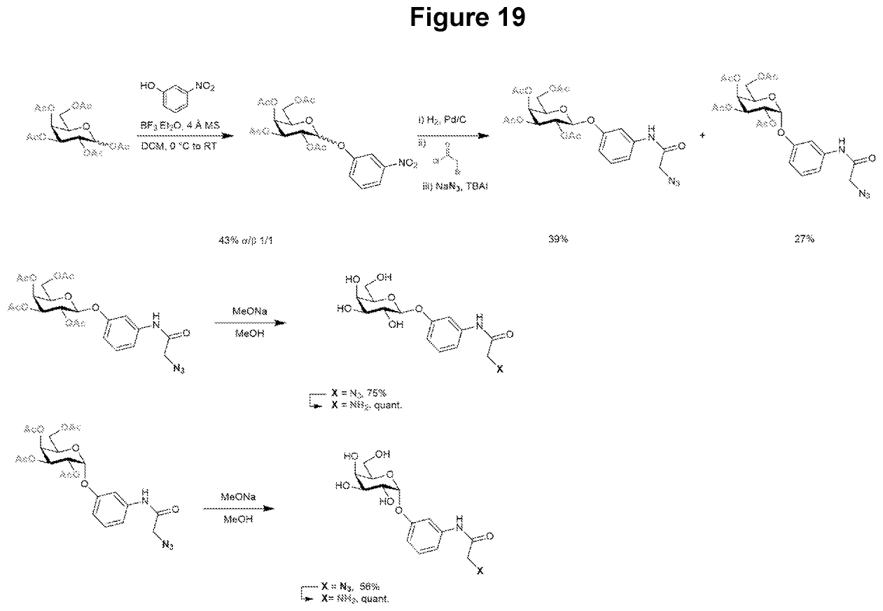

Suitably, the linker is a meta azido amido phenolate, wherein Z=NH₂ or N₃ and wherein X=O and A and C═H (see e.g. FIG. 19, final product).

According to specific embodiments of the invention, the linker group may be the following structure:

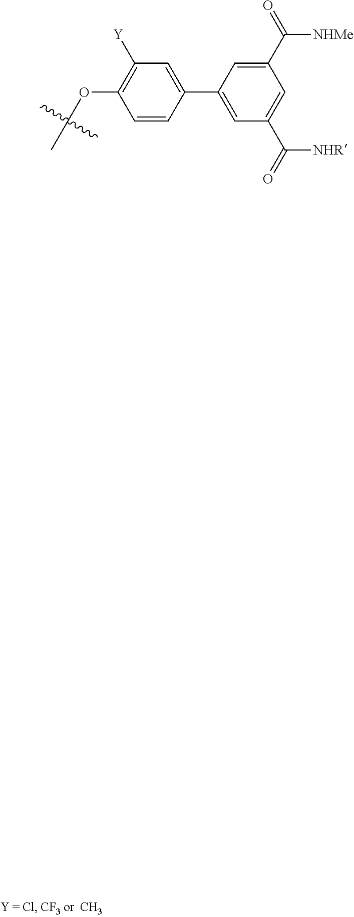

or combinations thereof.

According to specific embodiments of the invention, the linker group may be a 1,2,3-triazole form precursors such as 2-azidoethanol; 3-azidopropanol; 5-azidopentanol; 7-azidoheptanol; 10-azidodecanol. These linkers are particularly useful when click chemistry e.g. Huisgen reaction is used to connect the substrate to the glycan moiety which provides for a 1,2,3-triazole alcohol linker after the alkyne and the azide have been reacted.

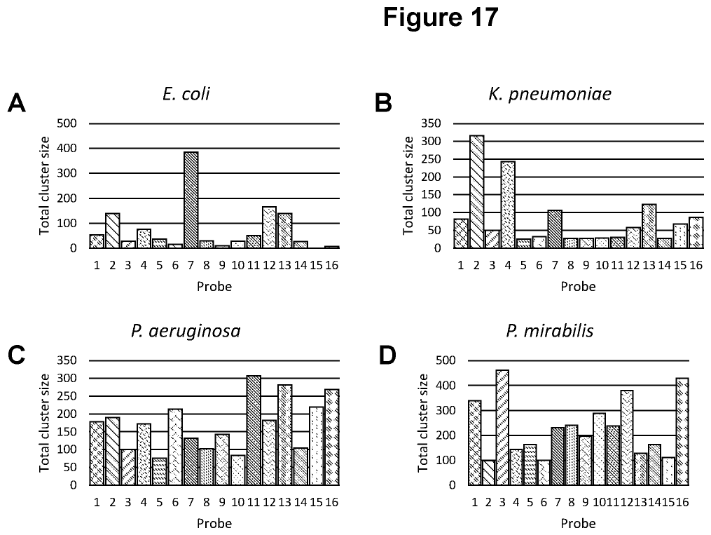

The configuration of the linker—such as its size and length—may be of particular importance to the functioning and specificity of the probe, as seen in the data presented for example in FIG. 17. When comparing C7-mannose to C3-mannose less efficient agglutination for E. coli can be observed. Despite the great affinity towards mannoside compounds an even bigger decrease in agglutination can be seen for C10-mannose. The attached glycan also plays a major role as when comparing the C7-mannose to the C7-xylose, no agglutination could be witnessed for C7-xylose.

The composition or probe, in specific embodiments of the invention, comprises at least one group that conforms to general Formula I, set out above and further comprises at least one group that conforms to general Formula II, set out below.

A—[B]ₙ II

wherein A includes a substrate tethered to the spacer group B of n atoms in length. Typically, A comprises a chemically functionalised surface such as a carboxylated or aminated surface which may be in particulate form, such as a microbead.

The length of the spacer group is such that n is suitably between about 5 and about 100 atoms; more suitably between about 6 and 80 atoms; more typically between about 7 and about 60 atoms; suitably between about 9 and about 40 atoms and most suitably between 30 and 40 atoms.

In embodiments of the invention, the spacer group may be a bifunctional polyether glycol (PEG) compound. The spacer group may be a bifunctional aminoalkyl ether compound, typically an aminoalkyl polyether compound, for example a C6, C8 or C10 aminoalkyl polyether compound.

Suitably the spacer group may be a bifunctional alkanolamine ether such 2-(2-(2-(2-aminoethoxy)ethoxy)ethoxy)ethanol or a bifunctional amine such as 4,7,10-trioxa-1,13-diaminotridecane. A number of spacers are described in Stephen A. Hill et al. Nanoscale, 2016, 8, 18630-18634, which is incorporated herein by reference.

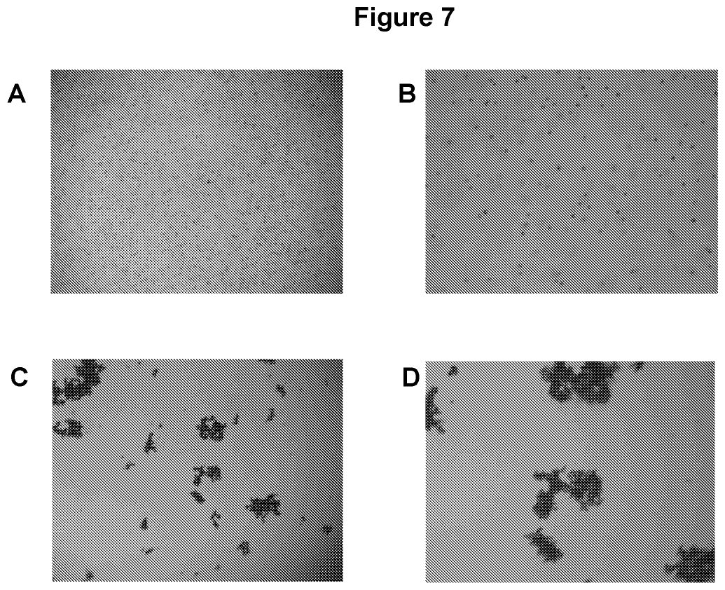

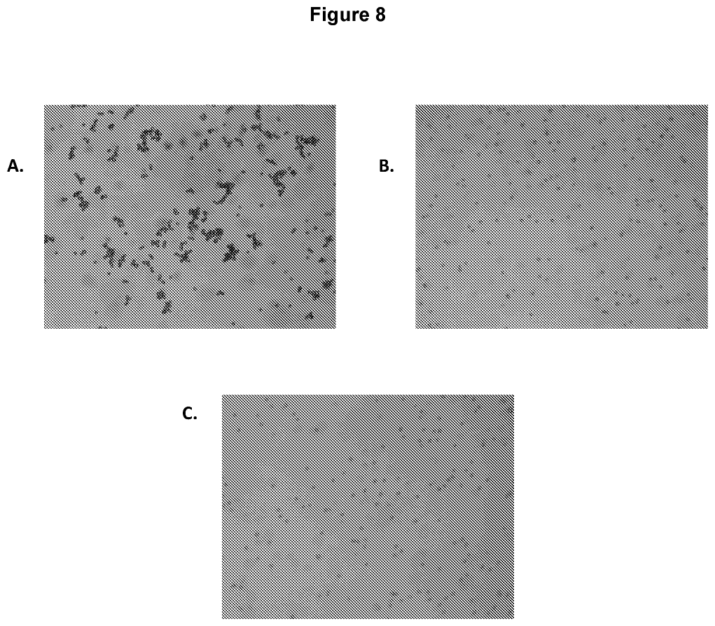

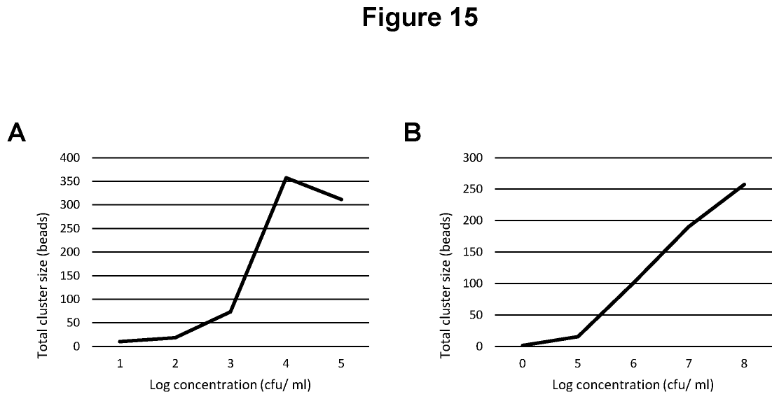

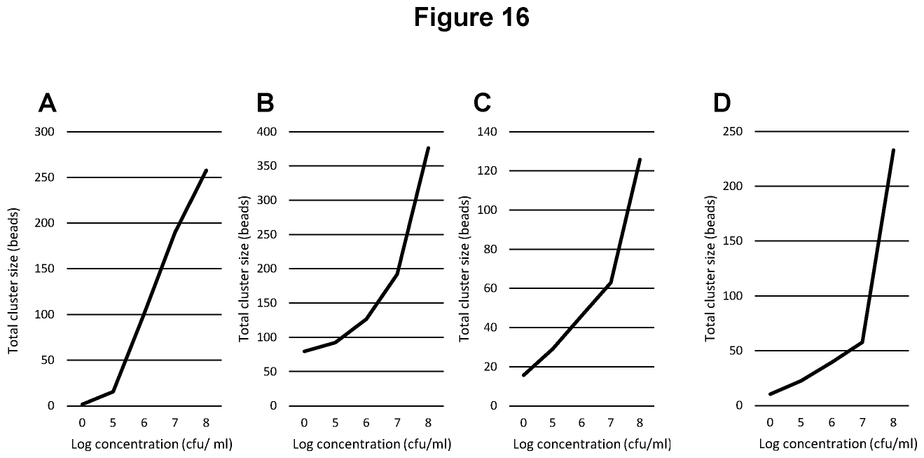

The presence of cellular material is detected by agglutination measurements (see FIGS. 7 and 8). The detection of cellular material can be qualitative and quantitative (see FIGS. 15 and 16). Qualitative detection is achieved by viewing probe sample mixtures on a light microscopy slide and determining, on the basis of the visible level of clumping or clustering, if agglutination has occurred. If clumping has occurred target cellular material must be present. Clumping is usually measured by Total Cluster Size (TCS). TCS is typically measured in number of beads.

In one embodiment of the invention, a quantitative detection is done by calculating the total cluster area in the sample as viewed under the light microscope. The clusters get bigger and are fewer as concentration of cellular material increases. Alternatively, light scattering techniques or fluorescence measurements may also be used to detect agglutination.

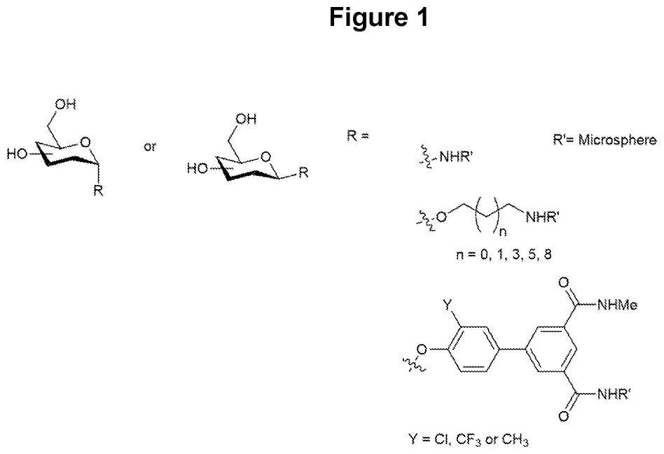

FIG. 1 is a scheme describing the nature of the linker. The linkers shown are bifunctional alkanolamine or alkanolamides such as 2-aminoethanol; 3-aminopropanol, 5-aminopentanol; 7-aminoheptanol; and 10-aminodecanol. The linkers may be coupled to the glycan as either the α- or β-anomer. As shown in FIG. 1 the substrate can also be directly coupled to the glycan without the need for a linker. In examples where the linker is not present the carboxylated substrate which may be a microbead, suitably made of latex is directly tethered to the aminated glycan.

The linker may be attached to the substrate by peptidic coupling or triazole formation through a 1,3-Huisgen cycloaddition. The linker may be a bifunctional linear alkanolamine, alkanolamide or a phenolic amide, typically a phenolic amide having a biaryl moiety such as of the formulae below.

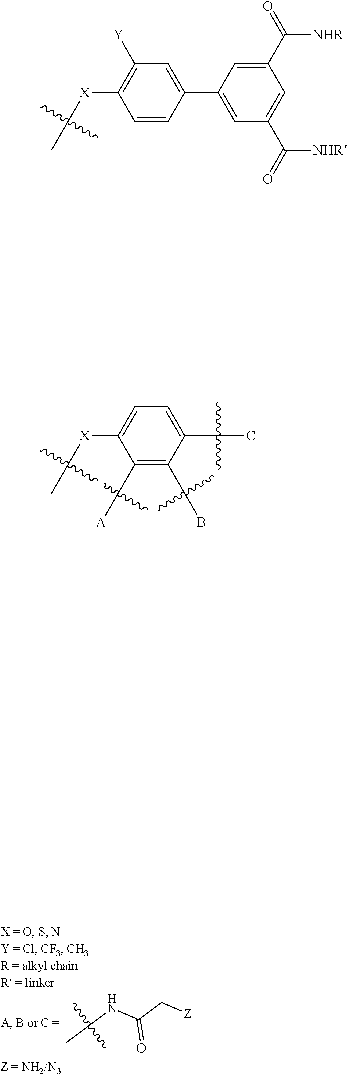

For example, the linker may be of the following structure:

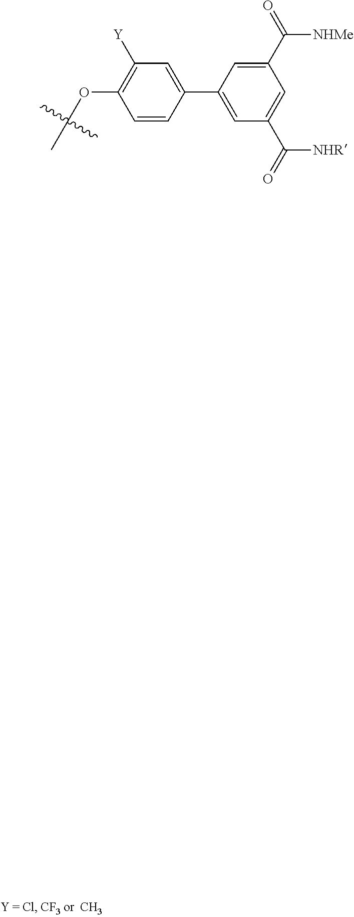

The composition may comprise a plurality of linkers which may be selected from the group consisting of bifunctional alkanolamine and alkanolamides such as 2-aminoethanol; 3-aminopropanol; 5-aminopentanol; 7-aminoheptanol; 10-aminodecanol; phenolic amides, typically a phenolic amide having a bis-aryl moiety or combinations thereof.

Typically the substrate is functionalised with a carboxyl moiety which can be attached to amine functionality by peptide coupling. Alternatively, the substrate may be functionalised with an amine moiety which can be attached to the carboxyl functionality of the linker by peptide coupling.

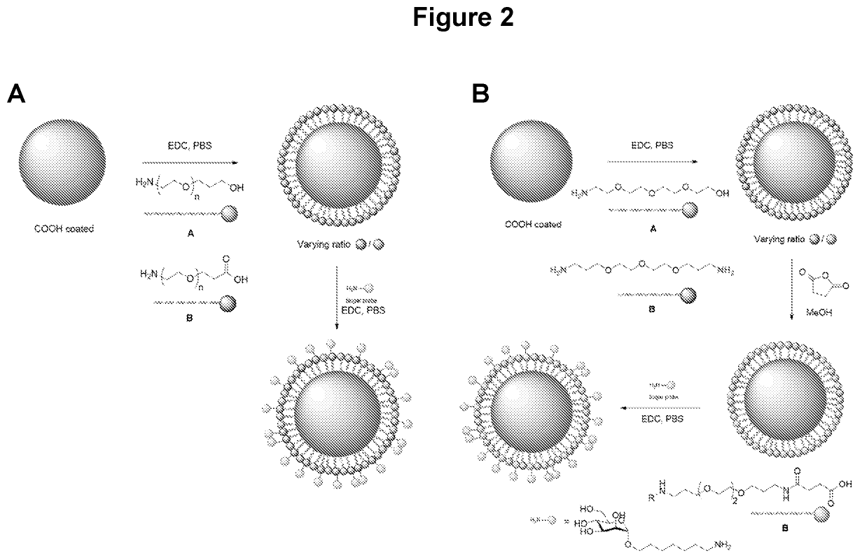

FIG. 2 is a scheme describing the functionalisation of the chemically modified substrate. The substrate is functionalised with linkers and spacers using peptide coupling reactions. The terminal end of the linkers is then coupled to the glycan moiety by peptide coupling using EDC in this example for both reactions. The ratio of linker and spacer can be varied to affect the sensitivity of the agglutination assay.

The spacers are bifunctional ether amino alcohols (FIGS. 2A and 2B; structure A) and may be selected from the group consisting of: C6, C8 or C10 aminoalkyl poly ether compounds (FIG. 2B). Suitably the spacer group is a bifunctional alkanolamine ether such 2-(2-(2-(2-aminoethoxy)ethoxy)ethoxy)ethanol.

Generally, the linkers are bifunctional ether amino acids (FIG. 2A, structure B) selected from the group consisting of: ether amino alcohols, amino alcohols or amino acids or combinations thereof.

Typically, the linker is a bifunctional diamine compound such as 4,7,10-trioxa-1,13-diaminotridecane which is further carboxylate to give a bifunctional ether amino acid so that the linker can be coupled to the aminated glycan as shown in FIGS. 2A and 2B, structure B.

Suitably the linker is of the following structures:

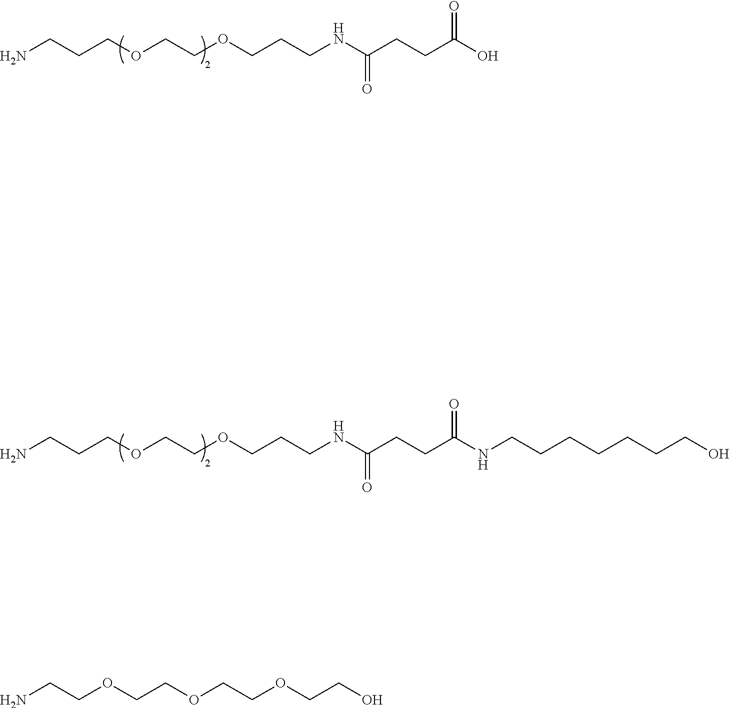

or combinations thereof.

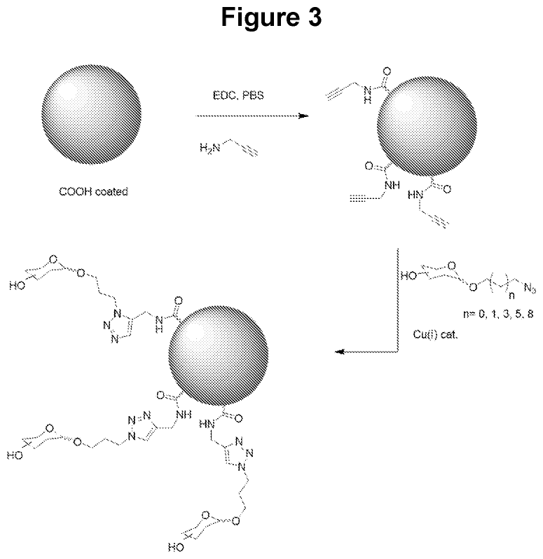

FIG. 3 is a scheme describing an alternative functionalisation of the chemically modified substrate e.g. a micro bead. The substrate is functionalised with bifunctional linkers such as amino alkynes or carboxyl alkynes and glycan moieties using peptide coupling reactions and Huisgen reactions. More specifically, the alkyne linker is attached to the substrate using a peptide coupling reaction. The terminal end of the alkyne linker is then coupled to the azidated glycan moiety by the Huisgen reaction using a copper(I) catalyst.

Typically, the linker is selected from the group consisting of: 3-amino-1-propyne; 3-carboxyl-propyne, azide alcohols, the following structure

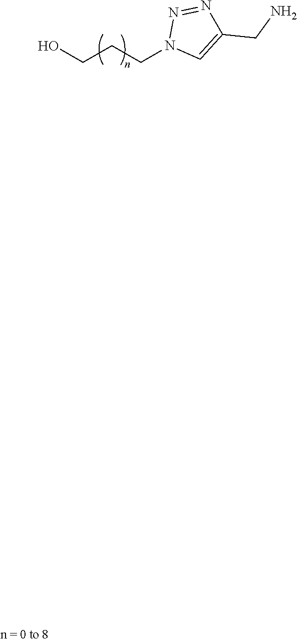

or combinations thereof.

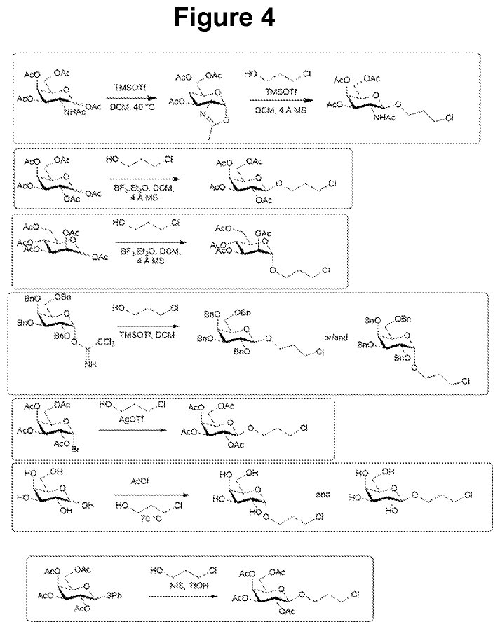

FIG. 4 is a scheme showing glycosylation reactions commonly used to install the linker on the anomeric position for the O-glycoside derivatives. Different glycosylation reaction conditions are used to direct the selectivity towards the desired outcome, α or β. The conditions also change depending on the glycan used.

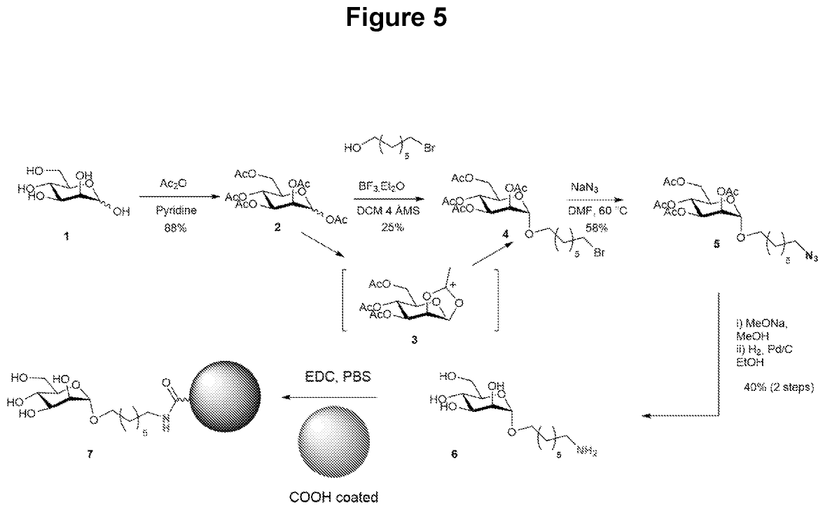

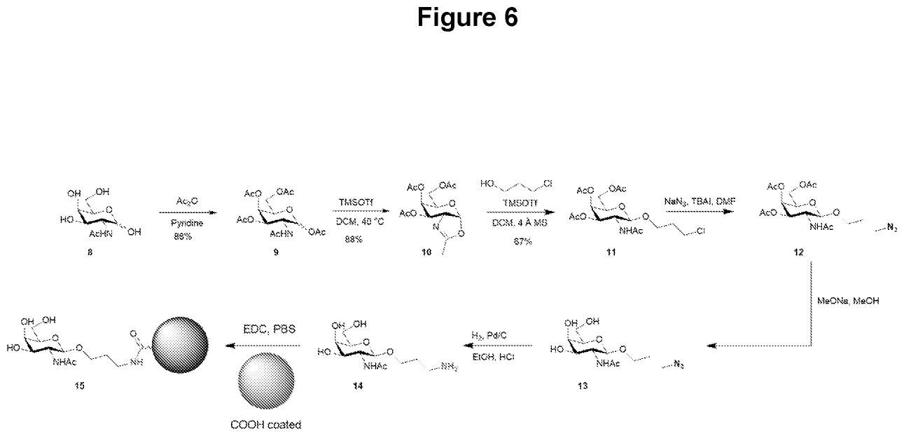

FIG. 5 is a scheme showing a route to a fully functionalised probe via glycosylation. Generally, the glycosylation reaction occurs early stage so that the linker can be installed selectively. The glycosylation reaction installs the desired anomer either selectively or a separation is required afterwards to isolate the desired α or β anomer (see Example 1 below for further detail). FIG. 6 is a scheme showing an alternative route to a fully functionalised probe via glycosylation (see Example 1 below for further detail).

FIG. 7 shows microscope images of samples containing the micro bead probe only (FIGS. 7A and 7B) and samples containing bacteria bound to the micro bead probe (FIGS. 7C and D). Clumping of the micro bead (agglutination) was clearly visible in samples with bacteria (FIGS. 7C and 7D) and absent in samples without bacteria (FIGS. 7A and 7B). FIGS. 7A and 7C show light microscope images at four times magnification. FIGS. 7B and 7D show light microscope images at ten times magnification. The bacterial sample contained P. mirabillis at a concentration of 10⁹ cfu/mL (colony forming units per millilitre). In all cases the xylose probe of Example 2 below was used.

FIG. 8 shows microscope images of samples containing different microorganisms. FIG. 8A shows P. mirabilis bound to the micro bead probe at a concentration of 10⁶ cfu/mL. FIG. 8B shows E. coli bound to the micro bead probe at a concentration of 10⁶ cfu/mL and FIG. 8C shows K. pneumoniae bound to the micro bead probe at a concentration of 10⁶ cfu/mL. In all cases the xylose probe of Example 2 below was used. In FIG. 8A the clumps indicative of agglutination is lesser and smaller than in FIGS. 3B and 3D since the bacterial concentration is lower. FIGS. 8B and 8D show no clumping/agglutination highlighting that the xylose based probe is selective for P. mirabilis. Agglutination was only exhibited in P. mirabilis (8A), albeit to a lesser extent than in FIGS. 7C and 7D since the bacterial concentration is lower. Meanwhile no agglutination was demonstrated in E. coli (8B) and K. pneumoniae (8C), highlighting that the xylose based probe is selective for P. mirabilis.

FIG. 15 is a line graph and shows dilution series or concentration gradient of: E. coli conjugated to the C7-mannose probe using EDC peptide coupling (A) and E. coli conjugated to the C7-mannose probe using CuACC click chemistry (B). The increase of total cluster size (TCS) with increase in bacterial concentrations shows that the probes are binding effectively in a concentration dependent manner meaning that the probes do not only detect bacteria through binding, but they are also able to quantify the amount of bacteria in the sample (see also FIG. 16 for further bacterial species). Expanding upon this further, FIG. 16 shows a relationship between a concentration gradient of E. coli and C7-mannose (A), K. pneumoniae and C₁₀-Mannose (B), P. aeruginosa and C3-Fucose (C), and P. mirabilis and C3-GlcNAc (D). The same concentration dependent agglutination response is demonstrated, meaning detection and quantification is also possible with the aforementioned combination of probe and bacterial species. FIG. 17 is a bar graph and shows total cluster sizes (TCS) as a measure for specificity of various probes for E. coli (A); K. pneumoniae (B); P aeruginosa (C); and P. mirabilis (D). The type of probe used is provided in Table 1 below. The ‘C’ nomenclature indicates the number of carbons in the linker and the type of coupling chemistry used is described in brackets (EDC=peptidic coupling). The larger the cluster the more specific the probe is for a certain bacterial species. Variable agglutination responses are seen with different combinations of probe and bacterial species. For example, C7-Xylose agglutinates P. mirabilis to a TCS of 462. This is four times higher than C7-Xylose with P. aeruginosa, which was the next highest TCS signal. The difference in TCS signal allows for identification and quantification of numerous bacterial species by multiplexing with multiple probes.

TABLE 1

| Probe | |
|---|---|
| Number | Glycan probe |
| 1 | C2-lactose (C2-Lac) (EDC) |
| 2 | C3-Mannose (C3-Man) (EDC) |
| 3 | C7-Xylose (C7-Xyl) (EDC) |
| 4 | C10-Mannose (C10-Man) (EDC) |
| 5 | C3-aGM1 (EDC) |
| 6 | C3-Fucose (C3-Fuc) (Click) |
| 7 | C7-Mannose (C7-Man) (Click) |
| 8 | 1-Amino-Fucose (1NH₂-Fuc) (EDC) |
| 9 | 1-Amino-Galactose (1NH₂-Gal) (EDC) |
| 10 | C3-GalNac (Click) |
| 11 | C3-GlcNac (Click) |
| 12 | 1-Amino-Lactose (1NH₂-Lac) (EDC) |
| 13 | 2-Amino-Sialic Acid(2NH₂-Neu5Ac) (EDC) |
| 14 | 1-Amino-Sorbose (1NH₂-Sorb) (EDC) |
| 15 | 1-Amino-Xylose (1NH₂-Xyl) (EDC) |
| 16 | 1-NH₂-Gal-β-1-4-GlcNac (EDC) |

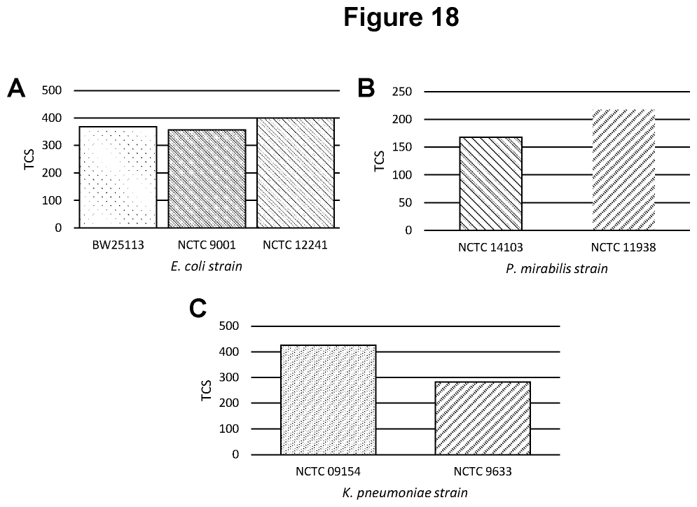

FIG. 18 is a bar graph and shows total cluster size (TCS) as a measure for specificity of various strains of the same species incubated with different probes: E. coli and C7-Mannose (A); P. mirabilis and C3-GlcNAc (B); and K. pneumoniae and C10-Mannose (C). Low variation in TCS signal is exhibited in different strains of the same bacterial species. This shows that the probes are specific to various strains of the same bacterial species.

Mannose Probes

According to specific embodiment, the present invention provides a probe for detecting bacteria having type 1 fimbriae, wherein the probe comprises a substrate to which is bonded a molecule of Formula III:

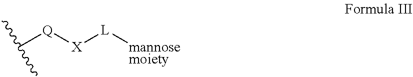

wherein: Q is a bond or a linker; X is other than hydrocarbylene and is of 1 atom in length; L is a hydrocarbylene group of 1 to 10 atoms in length; and the mannose moiety comprises at least one mannose residue.

The present invention can provide an easy to make and low-cost probe that can be used in a simple and reliable test for bacteria having type 1 fimbriae. In particular, the inventors have identified that the probes are particularly effective at detecting the bacteria when the mannose moiety has a glycosidic hydrocarbylene group of 1 to 10 atoms in length. In alternative embodiments the mannose moiety may be exchanged against other glycans which are described above under glycan probes.

The substrate is bonded by a molecule of Formula III. In other words, the substrate is functionalised by a molecule of Formula III. Typically, the substrate will be bound by molecules of Ill, i.e. multiple molecules of Formula III. The substrate may be bound by molecules that have different chemical structures that fall within the definition of Formula III. Suitably, the substrate will be bound by molecules that have the same chemical structure. Suitably, the molecule or molecules of Formula III are covalently bound to the substrate. By ‘the molecules of Formula III’, we are typically referring to substantially all molecules of Formula III that are bound to the substrate, or all molecules of Formula III that are bound to the substrate.

Group Q

Q in Formula III can be a bond or a linker. A linker can be used, for example, when surface residues of the substrate are incompatible with direct coupling to the remainder of the probe. For example, if the substrate has surface amino groups and the remainder of the probe is to be coupled at an amino group, a linker can be used to couple the two amino groups together. For example, a dicarboxylate can be used as the linker, whereby both amino groups can be coupled to the carboxylates using a peptide bond forming reaction. A linker can also be used to enhance surface passivation properties, which is particularly relevant when the substrate is a fluorescent dot such as a fluorescent carbon dot. For example, a well-known passivation agent that can be used as a linker is 4,7,10-Trioxa-1,13-tridecanediamine (TTDDA).

It is particularly preferred that the linker is a straight chain linker. The linker can be alkyl or alkyl substituted by one or more —O— or —NH— groups. Suitable substitutions are those which are stable. Stable substitution patterns will be known to the skilled person and will typically exclude, for example, —O—O— groups, —O—CH₂—O— groups, —O—NH— groups, —O—CH₂—NH— groups and the like. In other words, any heteroatom substitutions in the alkyl chain will suitably have at least a C₂ alkyl unit between them. Suitably, the linker is 1 to 30, 1 to 20, 1 to 15, 1 to 10, or 1 to 5, atoms in length. It is preferred that the straight chain linker lacks side groups containing more than 20, 15, 10, 9, 8, 7, 6, 5, 4, 3 or 2 atoms. It is particularly preferred that the straight chain linker lacks side groups.

In one embodiment, the linker comprises the formula —C(O)—Y—C(O)—, wherein Y is an optionally substituted alkyl, optionally substituted cycloalkyl, optionally substituted alkenyl, optionally substituted alkynyl, optionally substituted aryl, optionally substituted heteroaryl and optionally substituted heterocyclyl.

Group Y typically has a low molecular weight, for example, less than 1000, 900, 800, 700, 600, 500, 400 or 300 Da. Suitably, Y has a molecular weight of less than 500 Da, more suitably less than 400 Da, yet more suitably less than 300 Da.

Y can have the formula (—Z—)ₙ, wherein each —Z— is independently chosen from —CR²²—, —O—, or —NR²—, wherein each R² is independently selected from —H, —OH, —NH₂ or —C₁₋₆ alkyl, and n is from 1 to 30. Suitably, n is from 1 to 20, 1 to 15, 1 to 10, or 1 to 5. In one embodiment, R² is —H. In a particularly preferred embodiment, Y is (CH₂)2 (i.e. succinate) or NH(CH₂)₃O(CH₂)₂O(CH₂)₂O(CH₂)₃N (i.e. TTDDA).

Where Q is a linker, it is typically a low-cost and low molecular weight linker. This provides a substrate bearing a coating of low molecular weight molecules that is low-cost and stable for prolonged periods at room temperature. In addition to being low-cost and stable, the use of such a probe has been shown still to provide a highly effective probe.

Alternatively to being a linker, Q in Formula III can be a direct bond. Where Q is a direct bond, X is typically a functional group of the surface of the substrate. In other words, L can be directly bonded to the surface of the substrate. This makes for a particularly low molecular weight molecule according to Formula III that still provides for a highly effective probe.

Group X

According this aspect of the invention, X as used in Formula III is other than hydrocarbylene and is of 1 atom in length. In other words, X marks the end of the hydrocarbylene group of L. By 1 atom in length, we are referring to the length of the chain of atoms between the mannose moiety and the substrate. X may have further atoms appended to it, as long as those atoms do not form part of the length between the mannose moiety and the substrate. Where X has further atoms appended to it, it is preferred that no more than 6 atoms are appended. Where X has further atoms appended to it, suitably it is only the minimum number of atoms to fulfil valency requirements. Suitably, the appended atoms are selected from ═O, —OH and —H in accordance with valence and stability requirements.

In a preferred embodiment, X is selected from —C(O)—, —O—, —NR¹—, —S—, —S(O)—, —S(O)(O)—, or —P(O)OR¹—, wherein each R¹ is independently selected from H or C₁₋₆ alkyl. It is particularly preferred that X is selected from —C(O)—, —O—, or —NH—.

As mentioned, where Q is a direct bond then X is typically a functional group of the surface of the substrate. For instance, the surface of the substrate may be amino functionalised, in which case X will be —NR¹—, or the surface of the substrate may be carboxylate functionalised, in which case X will be —O—.

Alternatively, Q can be a linker. In this case, X will be other than hydrocarbylene and of 1 atom in length, and Q will correspond with this. For example, where Q is formed from succinate connecting two amino groups, Q will be —C(O)—CH₂—CH₂—C(O)— and X will be —NR¹—.

Group L

L in Formula 1 is a hydrocarbylene group of 1 to 10 atoms in length. By hydrocarbylene, we are referring to hydrocarbon groups such as alkyl, alkenyl, alkynyl and aryl groups.

By length, we are referring to the atoms in the chain between the mannose moiety and the substrate. Further hydrocarbylene side groups may be present. Where there are different options for following the chain length between the mannose moiety and the substrate (for example, the chain contains a cyclopentyl group), the shortest chain length should be counted. It is preferred that L has a molecular weight below 500, 400 or 300 Da, and suitably below 200 Da.

In a preferred embodiment, L is of formula (CR₂)ₙ, wherein n is 1 to 10 and each R is independently selected from:
H or C₁₋₆ alkyl, alkenyl, alkynyl, cycloalkyl or aryl; and/or
R₂ of a CR₂ is together an alkene of a C₁₋₆ alkyl, alkenyl, alkynyl, cycloalkyl or aryl; and/or consecutive CR₂ groups define an alkynyl group or alkenyl group, or part of a cycloalkyl or aryl group.

By this, we mean that L can comprise alkyl, alkenyl, alkynyl, cycloalkyl or aryl groups, or combinations thereof, in the chain between the mannose moiety and the substrate. In addition, side groups may also comprise alkyl, alkenyl, alkynyl, cycloalkyl or aryl groups, or combinations thereof.

Suitably, each R is —H and/or consecutive CR₂ groups define an alkynyl group, alkenyl group or aryl group.

In a particularly preferred embodiment, each R is —H. In other words, it is preferred that L is C₁₋₁₀ alkyl.

Where L is of formula (CR₂)ₙ, n can be at least 1, 2, 3, 4, 5, 6, 7, 8 or 9 and can up to 10, 9, 8, 7, 6, 5, 4, 3 or 2. Furthermore, n can be: 1 to 9, 1 to 8, 1 to 7, 1 to 6, 1 to 5, 1 to 4, 1 to 3, 1 to 2, 1, 2 to 10, 2 to 9, 2 to 8, 2 to 7, 2 to 6, 2 to 5, 2 to 4, 2 to 3, 2, 3 to 10, 3 to 9, 3 to 8, 3 to 7, 3 to 6, 3 to 5, 3 to 4, 3, 4 to 10, 4 to 9, 4 to 8, 4 to 7, 4 to 6, 4 to 5, 4, 5 to 10, 5 to 9, 5 to 8, 5 to 7, 5 to 6, 5, 6 to 10, 6 to 9, 6 to 8, 6 to 7, 6, 7 to 10, 7 to 9, 7 to 8, 7, 8 to 10, 8 to 9, 8, 9 to 10, 9, or 10.

In a preferred embodiment, n is 3 to 9, suitably at least 4, more suitably 4 to 8. In a particularly preferred embodiment, n is 7.

The inventors have identified that having a hydrophobic region at the mannose glycosidic position (i.e. hydrophobic L) provides for particularly effective probes. In particular, the use of such a hydrophobic region allows for low molecular weight probes that are easy and cost effective to manufacture in a highly reproducible manner.

Mannose Moiety

The mannose moiety may be exchanged against other glycans as described under glycan probes above. In cases where a mannose moiety is used, the mannose moiety comprises one or more mannose residues in a configuration suitable for binding to FimH of type 1 fimbriae. It has been identified that FimH binds to clusters of 3 mannose residues or to certain mannose moieties comprising multiple mannose residues, such as tri-mannose [Huang et al, Experimental Biology and Medicine 2016; 241: 1042-1053]. In a preferred embodiment, the mannose moiety is selected from mono-mannose or a polysaccharide comprising at least one mannose residue. Suitably the polysaccharide comprises at most 15, 10, 8, 7, 6, 5, 4, 3 or 2 mannose residues. It is preferred that the polysaccharide comprises at most 20, 15, 10, 8, 7, 6, 5, 4, 3 or 2 sugar residues. Suitably, the polysaccharide consists of mannose residues (i.e. the polysaccharide comprises only mannose residues and no sugar residues other than mannose). The polysaccharide may be linear or branched. The polysaccharide is suitably selected from Manα6[Manα3]Manα6[Manα3]-ManαO—, Manα6[Manα3]Manα6[Manα2-Manα3]ManαO—, and the trisaccharide Manα3-Manβ4GlcN—.

In a particularly preferred embodiment, the mannose moiety is mono-mannose. This is a particularly low molecular weight and cost-effective moiety, which has been demonstrated by the inventors as providing for an effective probe. When the mannose moiety is mono-mannose, the molecules of Formula III are suitably bound to at least 60%, at least 70%, at least 80%, at least 95% or at least 99% of the substrate surface, suitably 100% of the substrate surface.

It is preferred that the mannose moiety is attached to L by a glycosidic heteroatom. In a particularly preferred embodiment, a mono-mannose moiety is attached to L by its glycosidic heteroatom. The natural glycosidic heteroatom is —O—, but the glycosidic heteroatom can be manipulated. In a preferred embodiment the glycosidic heteroatom is —O— or —NH—. It is particularly preferred that the glycosidic heteroatom is —NH—.

The inventors have identified that the combination of mannose moiety and group L provides for a particularly easy to make, reproducible, stable and low cost probe that is highly effective in detecting bacteria. It is particularly surprising that a low molecular-weight molecule, having only a single mono-mannose, is highly effective in detecting bacteria.

Substrate

The substrate is any suitable substrate for making a probe. Whereas the mannose moiety and L are responsible for binding the bacteria, the substrate is generally responsible for transducing the binding into an observable signal.

The molecules of Formula III are suitably bound to 40 to 100% of the substrate surface. By this, we mean that of the surface functional groups available for binding, 40-100% of these functional groups are bound to molecules of Formula III. In one embodiment, the molecules of Formula III are bound to at least 50%, at least 60%, at least 70%, at least 80%, at least 90%, at least 95% or at least 99% of the substrate surface. Binding to 100% of the substrate surface is preferred where maximum concentration of mannose on the probe is desired. Binding to a lower percentage of the substrate surface may represent a balance between effectiveness of the probe and cost. This may be, for instance, where the molecules of Formula III are bound to 40% to 80%, suitably 50% to 70% or more suitably about 60% of the substrate surface.

The substrate is suitably a particle. It is particularly preferred that the particle is spheroidal. By spheroidal, we mean that the particle may have a rough and/or lumpy surface but overall approximates the shape of a sphere. The particle suitably has a diameter of 0.1 nm to 100 μm.

Where the particle has a rough and/or lumpy surface, the diameter is the average diameter (i.e. volume-based particle size, or diameter of the sphere that has the same volume as the particle).

A substrate that is a particle can provide an observable signal of binding in a variety of ways. For instance, the particle may give an optical readout to allow for optical detection of the probe binding to a bacterium. In a particularly preferred embodiment, the particle is fluorescent to give a fluorescent readout. By utilising a fluorescent particle bound to a mannose moiety, clustering of fluorescence around bacteria can be detected without any need for further conjugation of fluorescent imaging agents.

In another embodiment, the substrate may be of a size suitable for visualisation under a microscope (i.e. a diameter above 1 um). That is, clustering of probe and bacteria is observable without the need for fluorescence, although detection of a fluorescent readout may in some cases improve the accuracy of the reading.

In a further embodiment, the substrate may be of a size and coating density suitable for binding multiple bacteria, which in turn bind multiple further probes. This causes precipitation, or agglutination, of clusters of bacteria and probe.

When the substrate is a particle, it is therefore preferred that the particle is fluorescent at particle diameters of less than 1 μm.

In a further embodiment, the substrate can be a sheet of material such that detection of the bacteria occurs on the sheet of material.

The probe comprises a substrate to which is bonded a molecule of Formula III. Suitably, the molecule of Formula III is bonded to the surface of the substrate. By surface of the substrate, we are referring functional groups at the surface of the bulk material of the substrate that are available for binding. In the context of a sheet of material, we are referring to the surface that is intended for contact with a test sample.

The surface of the bulk material of the substrate may or may not be activated by functional group interconversions. In one embodiment, the substrate comprises no further surface displayed saccharides. In one embodiment, the substrate comprises no further cell-recognition markers. In one embodiment, the substrate is only functionalised by molecules of Formula III, suitably by molecules having only a single chemical structure according to Formula III.

The invention therefore provides for a simple, reproducible and cost-effective way to functionalise substrates to generate a stable and reliable probe for highly effective detection of bacteria.

Fluorescent Substrate

As mentioned, in a preferred embodiment the substrate is fluorescent. In other words, the probe is fluorescent. An ideal probe for bioimaging applications will have either a high quantum yield in the blue, or adequate green to red emission.

The probe may have an absorbance peak of at least about 350 nm. More suitably, the probe may have an absorbance peak of at least about 360 nm, 370 nm, 380 nm, 390 nm or 400 nm. Most suitably, the probe may have an absorbance peak of at least about 395 nm, 400 nm, 405 nm or 410 nm. The probe may have an absorbance peak of at most about 500 nm, 490 nm, 480 nm, 470 nm, 460 nm or 450 nm. Most suitably, the probe may have an absorbance peak of at most about 455 nm, 465 nm, 475 nm or 585 nm. For example, the absorbance peak may be at least about 400 nm and at most about 470 nm. The maximum absorbance peak wavelength may be at most about 460 nm, suitably 455 nm and typically 450 nm. The maximum emission peak wavelength may be any of, but not limited to: around 400 nm, between 395 nm-405 nm, at least about 400 nm, or 430 nm or 405 nm. The absorbance peak of the probe is substantially the same as the absorbance peak of the probe-bacteria complex.

The probe may have a fluorescence emission peak of at least about 350 nm. More suitably, the probe may have a fluorescence emission peak of at least about 360 nm, 370 nm, 380 nm, 390 nm or 400 nm. Most suitably, the probe may have a fluorescence emission peak of at least about 495 nm, 400 nm, 405 nm or 410 nm. The probe may have a fluorescence emission peak of most about 570 nm. More suitably, the probe may have a fluorescence emission peak of at most about 560 nm, 550 nm, 540 nm, 530 nm, 520 nm or 510 nm. Most suitably, the probe may have a fluorescence emission peak of most about 515 nm, 520 nm, 525 nm or 530 nm. For example, the fluorescence emission peak may be at least about 400 nm and at most about 520 nm. The maximum emission peak wavelength may be at most about 510 nm, suitably 515 nm and typically 520 nm. The maximum emission peak wavelength may be at least about 480 nm, suitably 490 nm and typically 500 nm. The fluorescence emission peak of the probe is substantially the same as the fluorescence emission peak of the probe-bacteria complex.

Fluorescent Dots

The particle of the probe can be a fluorescent dot, also known in the art as a fluorescent nanoparticle. Fluorescent dots are typically discrete, quasi-spherical or spherical nanoparticles, with sizes usually less than 10 nm diameter. In a preferred embodiment, the fluorescent carbon dots are at least 0.1, 0.2, 0.5, 0.8, 1, 1.5, 2, 3 or 4 nm in diameter and at most 10, 9, 8, 7, 6, 5, 4 or 3 nm in diameter. Specifically, the fluorescent dots can have a diameter of 0.1 nm to 10 nm, suitably 0.5 to 6 nm, more suitably 2 to 3 nm.

Suitably, the particle size of the probe (i.e. fluorescent dot plus molecule as shown in Formula III) determined by Atomic Force Microscopy (AFM) is in the ultrafine range. Specifically, the maximum average particle size of the probe may be at most about 50 μm. More suitably, the maximum average particle size may be at most about 40 μm, 30 μm, 20 μm, 10 μm, or 5 μm. Most suitably, the maximum average particle size may be at most about 10 μm, 9 μm, 8 μm, 7 μm, 6 μm, 5 μm, 4 μm or 0.1 μm. The minimum average particle size may be about 0.001 μm, 0.01 μm, 0.1 μm, 0.5 μm, 1 μm, 2 μm, or 5 μm. For example the average particle size may 0.01 μm (10 nm). Hence, in particular embodiments the invention includes utilisation of nanoscale FCDs with average particle sizes in the sub-micron range.

The fluorescent dot can suitably be a quantum dot (QD) or fluorescent carbon dot (FCD, sometimes also referred to herein as CD). Quantum dots are nanoparticles of semiconductor material, such as CdSe/ZnS, whereas fluorescent carbon dots are synthesised from suitable organic materials.

In general, it is preferred that the fluorescent dot is a fluorescent carbon dot. The advantage of fluorescent carbon dots over other fluorophores is that the core material is cheap to synthesize and stable to photobleaching and can be stored at room temperature for months (abolishing the need for a cold-chain), thus making them superior labels to alternative fluorophores [Hill and Galan, Beilstein J. Org. Chem., 2017, 13, 675-693]. FCDs are particularly suited to live cell imaging due to their water solubility, low toxicity and photo- and chemical stability. For example, FCDs have been used to visualise cells by cell uptake experiments [Hill and Galan, 2017].

The synthesis of FCDs is straightforward and has been reported elsewhere [see, for example, Hill and Galan, 2017; Hill et al., Nanoscale, 2016, 8, 18630-18634; Hill et al., Scientific Reports, 2018, 8:12234]. In one preferred embodiment, the fluorescent carbon dot comprises a glucosamine and m-phenylenediamine core.

FCDs typically make use of surface passivation agents (SPAs) for optimal photoluminescence properties and quantum yields. The mannose and L region (and optionally the Q region) of Formula III serve as both the SPA and for the enhanced bacterial detection properties.

Probes incorporating an FCD as the substrate can be used in the detection of bacteria by adding the probe to a sample and detecting fluorescence of the probe-bacteria complex. This can be done, for instance, by allowing the probe-bacteria complex to form, then washing away unbound probe, and then detecting residual fluorescence. Suitably, however, detection is performed by detecting clustering of fluorescence as multiple probes bind around the surface of a bacterial cell.

FCDs therefore represent a particularly effective substrate, as they provide for a probe that is low cost, easily and reproducibly manufactured, is stable in storage at room temperature for extended periods of time and is highly effective at detecting bacteria.

Microbeads

In one embodiment, the particle is a bead comprising a plastics material. Suitably, the plastics material is latex. In one embodiment, the bead is a fluorescent bead.

Suitable beads are known in the art and are commercially available. The beads suitably have a diameter of 0.1 to 1000 μm. In particular, the beads can have a diameter of at most 900, 800, 700, 600, 500, 400, 300, 200, 100, 80, 60, 50, 40, 30, 25, 20 or 15 μm. Suitably, the bead has a diameter of 1 to 50 μm, more suitably about 10 μm. These particles can also be referred to as microbeads, microparticles or microspheres. As set out above, beads with diameters greater than about 1 μm can be detected by microscopy without the need for a fluorescence readout. Furthermore, these larger particle sizes can lead to agglutination of the bacteria having type 1 fimbriae when such bacteria are present in a test sample.

The inventors have shown that bead substrates according to the invention are particularly effective in detecting bacteria having type 1 fimbriae. In a preferred embodiment, the bead used for formation of the probe is a surface-carboxylated bead.

Composition

According to another aspect, the present invention provides a composition comprising a probe according to any preceding claim and a buffer. The probe and buffer may be in the solid state.

In this case, the probe and the buffer would typically be reconstituted in solution, suitably aqueous solution, prior to use in detection of bacteria. In a preferred embodiment, the composition is in solution, suitably aqueous solution.

Certain fluorescent materials perform optimally at specific pH values. As such, the choice of buffer and quantity of buffer are typically selected to maintain the pH at the optimal value for optimal fluorescence. The concentration of buffer should be sufficient to maintain the pH when the probe is mixed with the test sample.

Use

According a further aspect, the present invention provides for the use of a probe according to the first aspect of the invention or a composition according to the second aspect of the invention for the detection of bacteria having type 1 fimbriae. The probes of the invention can also be used as a fluorescence label when observing a mixed culture.

While the probe of the invention will typically be used for analysing the presence of bacteria having type 1 fimbriae optically, it is also possible that the probe can be used to capture the bacteria. The probe will bind to and retain the bacteria while the remaining test sample is washed away. Then, the bacteria can be released from the probe. In particular, the bacteria can be released by adding a mannose solution in sufficient concentration to displace the probe from the FimH adhesin. Any suitable material can be used for the substrate as long as it allows for separation of the probe-bacteria complex from the test sample. For example, the probe can be solid and of a certain size to allow for separation of the test sample by filtration. Equally, the probe could be used as a chromatography stationary phase and the bacteria eluted with a mannose solution. Other materials for the substrate include a magnetic substrate for separation by magnetism, or a substrate that comprises further affinity markers for purification by appropriate affinity catch-and-release purification systems. Such systems are known in the art.

Methods of Detection

The present invention provides a method of detecting bacteria having type 1 fimbriae in a test sample, the method comprising the steps of:
a) providing a probe according to the first aspect of the invention or a composition according to the second aspect of the invention;
b) contacting the probe or composition with a test sample;
c) providing sufficient time for the probe to bind to the bacteria; and
d) detecting the presence of the probe-bacteria complex.

In a preferred embodiment, the test sample is an isolated body fluid, isolated tissue sample, foodstuff, or surface swab. It is particularly preferred that the test sample is a urine sample.

The detection methods of step d) are not particularly limited. For instance, the probe-bacteria complex may be separated and/or purified from the test sample and analysed in a further step for the presence of bacteria.

While it is possible that detection can be carried out using further test materials, such as antibody tests or culture growth tests as are known in the art, a significant advantage of the invention is that the probe can allow for detection without the need for purification steps or further test materials. For instance, when bacteria having type 1 fimbriae are present, the probe can bind to the bacteria and create specific patterns such as agglutinated clusters or the formation of fluorescent outlines of bacterial cell shapes or fluorescent bright spots representing multiple fluorescent probes gathering around a bacterial cell. As such, in a preferred embodiment of the invention, step d) further comprises detecting the formation of a pattern indicative of a probe-bacteria complex. This step can be conducted without separating the probe-bacteria complex from the test sample. Furthermore, this step can be conducted without separating the probe-bacteria complex from unbound probe. These detection methods are set out in further detail below.

Probe-Bacteria Clusters (Optical or Fluorescence Enhanced)

In a preferred embodiment, the substrate is a particle and step d) comprises the detection of clusters of probe-bacteria complex. In this embodiment, the particle is capable of causing agglutination. By this, we mean that the probe and bacteria bind together to create visible clusters. Typically, the particle is a microbead. Such microbeads generally have a larger surface area than the bacteria, which mimics conditions in the host body during infection. The larger surface area coupled with shear force favours adhesion of the bacteria to the probe. When a bacterial cell attaches to two or more probes, the probe-bacteria complexes clump together, hence the term agglutination, to form a cluster. In this detection method, therefore, the microbead will typically have a diameter of at least 1 μm, suitably at least 2 μm, more suitably at least 5 μm, in order to facilitate cluster formation. In a preferred embodiment, the microbead has a diameter of 1 μm to 100 μm, suitably 2 μm to 50 μm, more suitably 5 μm to 20 μm. Suitably, the microbead has a diameter of about 10 μm. The microbead will suitably have molecules as shown in Formula III bound to 100% of the substrate surface.

As the microbead-based probes described herein specifically target the bacteria fimbriae, agglutination should only occur when the bacteria fimbriae are attached to the microbead-based probe. An important advantage is that the clusters of bacteria and probe are visible using simple bright-field microscopy. A further very important advantage arises from this: the unbound bacteria or probes do not need to be removed from the test sample ahead of the detection step. As such, the probe is simply mixed with the test sample a positive result is indicated by the formation of clusters. The formation of clusters can therefore be detected, for example, by bright-field microscopy. Other detection techniques include measuring optical density, light scattering or fluorescence. A cloudy sample (>10\hat{ }6 CFU) becomes less cloudy. Analysis can be done by eye or can be automated through use of software analysis. It is noted that some clusters can form in the absence of bacteria, simply by microbead-based probes settling by each other. This can be corrected for by running and comparing with a blank negative control. The blank can be conducted in parallel with the assay, or can be a pre-calibrated standard (particularly if software detection is used).

When software is used, the detector can be programmed to automatically analyse the image and then output whether the result is positive or negative without the need for operator intervention, obviating the need for training the operator in detection of clusters.

Significantly, by analysing for the formation of clusters, there is no need to separate or purify the probe-bacteria complex from the test substrate or the unbound probe. This leads to a significant enhancement in the speed of the analysis. This also removes the need for further handling steps, and therefore removes the need for training an operator in conducting those steps. The removal of further handling steps also removes any error associated with those steps, such as contamination, spillage, inadvertent switching or misplacement of samples, etc. To assist with visualisation of the clusters, the microbead can be fluorescent. However, this is not essential. When the microbead is fluorescent, the formation of clusters can be detected by bright-field and/or fluorescence microscopy. When fluorescence microscopy is used, there may be an additional fluorescent signal from fluorophores in the test sample. However, as the additional fluorophores are typically not able to bind to bacterial cells, such contaminants will generally not cluster around the bacteria. As such, even in the presence of fluorophore contaminants, the formation of clusters still allows for simple detection of a positive result without needing to separate or purify out the contaminants or unbound probe.

Fluorescence Detection

As set out above, when fluorescent substrate is used the fluorescence can be exploited to enhance the detection of clusters. In one embodiment, the substrate is a fluorescent dot, suitably a fluorescent carbon dot. Fluorescent dots are much smaller than microparticles. Owing to their size, probes that utilise a fluorescent dot as the substrate do not facilitate agglutination. Instead, when a bacterium having type 1 fimbriae is present, multiple fluorescent dot-based probes will adhere to the many type 1 fimbriae that surround a bacteria cell. This accumulation of fluorescent dots around each bacterial cell can be detected by fluorescence microscopy. In effect, the accumulation of fluorescent dots will have the effect of lighting up each bacterial cell to which the probe adheres. Depending on the level of magnification, this can be detected by looking for fluorescent bacterial cell outlines forming, or at lower magnifications by looking for the formation of bright spots of fluorescence. By looking for the formation of these specific patterns, a positive result can be determined without needing to separate or purify the probe-bacteria complex from the test sample or unbound probe. For instance, physiological samples such as urine are complex systems potentially containing a variety of microorganisms, proteins, hormones, urea, various metabolites and compounds such as riboflavin which may have fluorescent properties. Using probes according to the present invention, the probe-bacteria complex does not need to be separated from the test sample, such as a physiological sample, for detection. As such, test samples can be analysed for the presence of bacteria having type 1 fimbriae using a rapid, easy-to-use and reliable method.

Fluorescence Detection—Multi-Wavelength Analysis

In a preferred embodiment the substrate is fluorescent and step d) comprises sequentially exciting the substrate using at least two different peak emission wavelengths and detecting the fluorescence signal. Typically, irradiation and detection will be carried out using a spectrofluorometer. In other words, this method will typically involve recording the total fluorescence output from a sample rather than a pattern recognition analysis. In this method, the probe-bacteria complex will typically have undergone the additional step of separation from the test sample and/or the unbound probe. However, this detection method may be used in combination with pattern recognition to further improve accuracy.

As set out in the applicant's earlier application WO-2021/038515, which is incorporated herein by reference, excitation at one or more wavelengths across the absorbance spectrum of the probe or test sample allows for efficient detection of bacteria in the presence of additional naturally present fluorophores, therefore eliminating false positive results. The probes of the present invention, wherein the substrate is fluorescent, are particularly effective when deployed with this detection method, providing for an easy-to-use, rapid and reliable method for detection of bacteria having type 1 fimbriae.

Using different excitation wavelengths within the absorption spectrum of the probe fluorophore improves the ability to determine if the fluorophore is present. There may be different ways of determining this including, but not limited to, comparing the light emitted from the fluorophore after being excited by the different wavelengths and seeing whether the different detected intensities correspond to the expected wavelength absorption profile of the fluorophore.

This measurement and determination capability may be improved by including three or more excitation wavelengths, for example, including a light source for each of the different excitation wavelengths. These light sources may be chosen to have peak emission wavelengths within one of the tails of the excitation spectrum of the probe fluorophore. Excitation wavelengths chosen along a tail of the excitation spectra typically produce measurably different light emissions from the target fluorophore. The more excitation wavelengths used across the width of the target excitation tail, the greater the ability to discern the presence of the target fluorophore and the presence of unwanted fluorophores.

Physiological samples such as urine are complex systems potentially containing a variety of microorganisms, proteins, hormones, urea, various metabolites and compounds such as riboflavin which may have fluorescent properties. Fluorophores with similar emission wavelengths to fluorescent probes, if present in large enough concentration, can potentially interfere with a fluorescence measurement. Excitation at multiple wavelengths is advantageous as it allows for the detection of another fluorophore which may be present in the physiological sample. In addition to sampling a fluorescence output at only one excitation wavelength at a time, sampling occurs at various wavelengths across the absorbance spectrum including the maximum peak absorbance wavelength. For example, the physiological sample may be excited at about 405 nm, 430 nm and 450 nm. LEDs are one example of a suitable light source. Other light sources, such as filtered white light sources, may be expensive. The sample may be excited by a first narrowband light source at least at about 360 nm, 370 nm, 380 nm, 390 nm or 400 nm. Most suitably, physiological sample may be excited by a first narrowband light source at least at about 395 nm, 400 nm, 405 nm or 410 nm. The physiological sample may be excited by a first narrowband light source at most at about 410 nm, 415 nm or 420 nm. Most suitably, the physiological sample may be excited by a first narrowband light source at about 405 nm. The light source may be an LED light source.

The sample may be excited by a second narrowband light source at least at about 390 nm, 400 nm, 410 nm, 420 nm or 430 nm. Most suitably, physiological sample may be excited by a second narrowband light source at least at about 395 nm, 405 nm, 425 nm or 435 nm. The physiological sample may be excited by a second narrowband light source at most at about 440 nm, 435 nm or 430 nm. Most suitably, the physiological sample may be excited by a first narrowband light source at 430 nm. The light source may be an LED light source.

The sample may be excited by a third narrowband light source at least at about 410 nm, 420 nm, 430 nm, 440 nm or 450 nm. Most suitably, physiological sample may be excited by a third narrowband light source at least at about 425 nm, 430 nm, 435 nm or 445 nm. The physiological sample may be excited by a third narrowband light source at most at about 480 nm, 455, 470 nm or 460 nm. Most suitably, the physiological sample may be excited by a first narrowband light source at about 450 nm. For example, the physiological sample may be exited at about 405 nm, 430 nm and 450 nm. The sample may be excited by multiple wavelengths simultaneously. The sample may be excited by multiple wavelengths consecutively. It is also desirable to choose excitation wavelengths for which narrowband sources are commercially available. The light source may be an LED light source.

Kit

The invention provides a kit comprising a probe as described herein, and an apparatus for contacting the probe or composition with a test sample. The apparatus can be a vessel, surface or other device suitable for contacting the probe with a test sample. Suitably, the apparatus will be compatible with a suitable detector. Suitably, the apparatus will also assist with the detection. For example, the apparatus can present the sample on an appropriate background for detection. The apparatus can, for example, be a test strip, flow device, cuvette or microtiter plate. In a preferred embodiment, the kit further comprises a detector for detection of the presence of the probe-bacteria complex. A detector may comprise optical apparatus such as a magnifier, a microscope or digital image analysis equipment.

Probe Concentrations

The ratio of probe to test sample is important for the labelling. If the probe concentration introduced is very small, not all bacteria in the sample may be labelled. If the concentration of probe in the test sample is very high, the sample will likely be saturated. Therefore, it is preferable for the probe concentration in the test sample to be at most 200 μg/mL. More suitably, the probe concentration in the test sample may be at most 190 μg/mL, 180 μg/mL, 170 μg/mL, 160 μg/mL, or 150 μg/mL. Most suitably, the probe concentration in the test sample may be at most 140 μg/mL, 130 μg/mL, 135 μg/mL, 133 μg/mL, 134 μg/mL, 120 μg/mL or 110 μg/mL. The minimum probe concentration in the test sample, at the point of detection, may be 0.01 μg/mL, 20 μg/mL, 30 μg/mL, 40 μg/mL, 50 μg/mL, or 60 μg/mL or 80 μg/mL. For example, 2 ml of 200 μg/mL probe may be added to a 3 mL (133 μg/mL) test sample such as a urine sample.

Embodiments described for the mannose probes above are generally applicable to all glycan probes described herein. All references cited herein are incorporated by reference in their entirety. Unless otherwise defined, all technical and scientific terms used herein have the same meaning as commonly understood by one of ordinary skill in the art to which this invention belongs.

### EXAMPLES

### Example 1—Probe Synthesis

Probe Synthesis Via Peptidic Coupling

A library of probes has been synthesised according to methods described in Benito-Alifonso et al. (Angew Chem Int Ed Engl. 2014 Jan. 13; 53(3): 810-814). However, instead of quantum dots latex microbeads have been used as the substrate.

The glycans were attached to the latex micro beads via a linker by peptidic coupling. FIG. 1 shows a number of linkers which have been investigated. See also FIGS. 2A and 2B for reference.

For example, glycosylamines were prepared by microwave assisted Kochetkov amination as per Bejugam et al. (Bejugam M, Flitsch SL. Org. Lett. 2004; 6:4001). 1.5 g (0.01 mol) of xylose in cases where the glycan is xylose was dissolved into 5 mL of an ammonia in methanol (7 M) solution. The high pressure vials were capped and the reaction was heated to 50° C. for 24 hours. Crystallization of amino xylose occurred in the vial after 1.5 g (0.01 mol) of xylose was dissolved into 5 mL of an ammonia in methanol (7 M) solution. The high pressure vials were capped and the reaction was heated to 50° C. for 24 hours. Crystallization of amino xylose occurred in the vial after synthesis. As a consequence the xylose at the bottom of the flask remained isolated from the ammonia solution despite the stirrer in the reaction vial. The crystals from the top of the reaction were washed with methanol using vacuum. 150 mg of amino xylose crystals (10% yield) were finally collected.

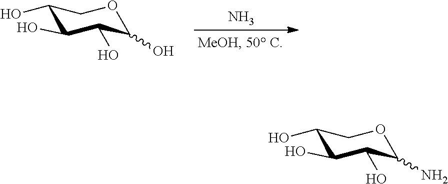

¹H NMR (400 MHz, DMSO-d6) δ 4.83 (dd, J=4.8, 3.3 Hz, 2H, —OH), 4.46 (d, J=4.1 Hz, 1H, —OH), 3.65 (d, J=8.4 Hz, 1H, C1), 3.55 (dd, J=11.1, 5.3 Hz, 1H), 3.20 (ddt, J=10.3, 8.7, 5.2 Hz, 1H), 3.02 (td, J=8.7, 4.5 Hz, 1H), 2.94 (dd, J=11.1, 10.4 Hz, 1H), 2.74 (td, J=8.6, 4.1 Hz, 1H)ppm; ¹³C NMR (101 MHz, DMSO-D6) δ 87.6 (C1), 77.8, 75.5, 70.5, 67.3 ppm.

The bifunctional linker was attached to the aminoglycosides as well as to the carboxyl functionalised microbeads using peptide coupling reactions via N-(3-dimethylaminopropyl)-N′-ethylcarbodiimide hydrochloride (EDC) as the coupling reagent (see FIG. 2).

Alternative probes using different sugars were synthesised as described in Carb. Res. 1995, 266, 211-219 and Org. Biomol. Chem., 2018, 16, 74307437.

Probe Synthesis Via Glycosylation

The glycan is attached to the chemically modified substrate by glycosylation followed by peptidic coupling as described in in FIGS. 4 to 6.

For example, in FIG. 5 starting from deprotected mannose 1 first an acetylation is necessary (an acetate group on position two is used as a directing neighbouring group for the glycosylation reaction). Peracetylated mannose 2 was then subjected to the lewis acid Bf₃·Et₂O to form the transient oxonium ion 3, which was then reacted with bromo-heptanol to give 4. Bromide 4 was then reacted with sodium azide, displacing efficiently the bromide and forming azide 5 efficiently. The last two steps were deprotection of the acetates followed by the reduction of the azide moiety to the desired amino intermediate. The final step was to link 6 to the beads through a peptidic coupling (FIG. 5, compound numbers relate to FIG. 5).

Similar to the synthesis scheme shown in FIG. 5, in FIG. 6 the first step is the peracetylation of e.g. GalNac 8 to give the protected glycan 9. The next step was the formation of the oxazoline intermediate 10. Opening the oxazoline with chloropropanol in presence of lewis acid TMSOTf afforded the desired β-glycoside 11 in high yield. Finally, treated chloride 11 with sodium azide and TBAl will afford the expected azide 12. Deacetylation with sodium methoxide followed by reduction of the azide moiety will afford intermediate 14 that will be conjugated to a probe via peptidic coupling (compound numbers relate to FIG. 6).

Probe Synthesis Via the Azide-Alkyne Huisgen Cycloaddition

In cases where the Huisgen reaction was used, the alkyne was attached to the micro bead or substrate with a peptidic coupling as described above. These types of reactions applicable to probe synthesis are described in Vida Castro, Hortensia Rodríguez, and Fernando Albericio, ACS Comb. Sci. 2016, 18, 1-14. Azide derivatives of different sugars where then coupled directly to the micro bead. A reaction scheme highlighting such a coupling via the Huisgen reaction is provided below (R′=microbead; see also FIG. 3).

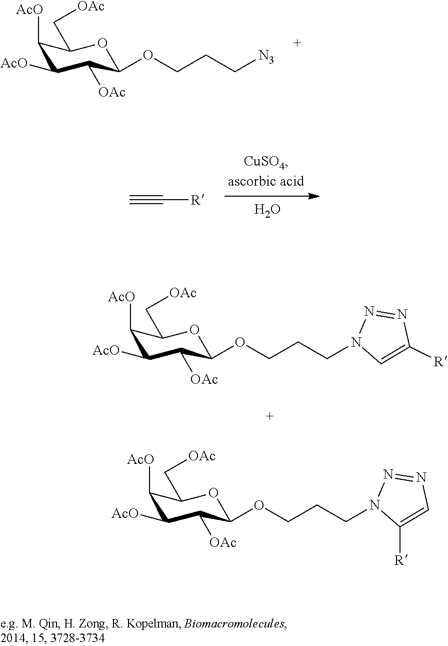

General Protocol for Immobilisation of Mannosides to CML Latex Beads Using EDC Coupling

The compound numbers used in the below synthesis protocols apply to the schemes shown for each compound.

500 μL of CML beads 4% w/v were centrifuged (8000 RPM, 20 min) supernatant was removed and replaced with PBS (500 μL). EDC (33 μL of a freshly prepared 19 mM solution in PBS) was added to the solution of beads then the RM was agitated for 10 min. The desired amino mannoside (25 μL of an 18 mM solution in H₂O/PBS ratio 5/3) was then added. The RM was then agitated 16 h then centrifuged (8000 RPM, 20 min), the supernatant removed and replaced with diH₂O (500 μL). The process was repeated 3 times then the supernatant was replaced with MeOH (500 μL), centrifuged (8000 RPM, 20 min), and the supernatant replaced with sterile PBS (500 μL) to provide a 4% w/v of conjugated beads.

General Protocol for Immobilisation of Mannosides to CML Latex Beads Using CuAAC

500 μL of CML beads 4% w/v were centrifuged (8000 RPM, 20 min) supernatant was removed and replaced with PBS (500 μL). EDC (33 μL of a freshly prepared 19 mM solution in PBS) was added to the solution of beads then the RM was agitated for 10 min. Propargylamine solution (25 μL of an 18 mM solution in H₂O/PBS ratio 5/3) was then added. The RM was then agitated 16 h then centrifuged (8000 RPM, 20 min), the supernatant removed and replaced with diH₂O (500 μL). The process was repeated 3 times then the supernatant was replaced with MeOH (500 μL), centrifuged (8000 RPM, 20 min), and the supernatant replaced with PBS (500 μL) to provide a 4% w/v of propargylated beads. A solution of azido mannoside (25 μL of a 0.56 mM solution in PBS) was added to the RM followed by a freshly prepared solution of ascorbic acid (63 μL of a 5.8 mM solution in PBS), placed under N₂ then shaken for 10 min. A CuSO₄ solution (50 μL of 1.43 mM) The RM was placed under N₂ atm then shaken for 16 h. The RM was then centrifuged (8000 RPM, 20 min), supernatant replaced with diH₂O (500 μL), this step was repeated 3 times then MeOH (500 μL) was added and the RM centrifuged (8000 RPM, 20 min), the supernatant was subsequently removed and replaced with sterile PBS (500 μL) to provide a 4% w/v of conjugated beads.

Glycan Synthesis Specific for E. coli

### 1,2,3,4,6-Penta-O-acetyl-D-mannopyranose

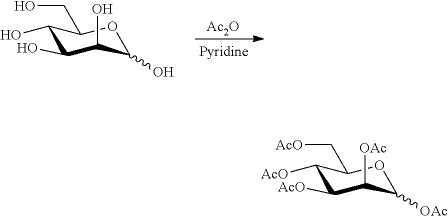

To a solution of D-mannose (10 g, 55.5 mmol) in pyridine (100 mL) was added acetic anhydride (100 mL). After 16 h the RM was quenched with water (100 mL), then diluted with DCM (100 mL) and washed with HCl 1 M (3×100 mL), HCl 5 M (100 mL) and then NaHCO₃ sat. (100 mL), dried over Na₂SO₄, filtered and concentrated in vacuo to give 1,2,3,4,6-penta-O-acetyl-_{D}-mannopyranose (19.1 g, 48.9 mmol, 88%, α/β: 1/2) as a clear oil.

¹H NMR (400 MHz, CDCl₃): δ 5.83 (1H, d, J 1.8 Hz, H-1β), 5.71 (1H, s, H-1α), 5.24 (1H, d, J 2.2 Hz), 5.10-5.09 (2H, m), 5.03-4.99 (2H, m), 4.08-4.01 (2H, m) 3.90-3.83 (3H, m), 3.68-3.64 (1H, m), 1.96 (3H, s, COCH₃), 1.94 (3H, s, COCH₃), 1.86 (3H, s, COCH₃), 1.84 (3H, s, COCH₃), 1.82 (3H, s, COCH₃), 1.76 (3H, s, COCH₃), 1.75 (3H, s, COCH₃) ppm; ¹³C NMR (101 MHz, CDCl₃): δ 170.3, 170.3, 169.9, 169.6, 169.5, 169.4, 169.3, 168.2, 168.1, 167.8, 90.3 (C-1β), 90.1 (C-1α), 72.8 (C-5α), 70.3(C-5β), 70.3, 68.8, 65.2 (C-2, C-3, C-4α), 68.5, 68.0, 65.2 (C-2, C-3, C-4β), 61.9 (C-6β), 61.8 (C-6α), 20.5, 20.5, 20.4, 20.4, 20.4, 20.4, 20.4, 20.3, 20.3, 20.2 ppm; HRMS(ES): calcd. for C₁₆H₂₂O₁₁Na 413.1054; found 413.1057. Spectral values were in accordance with those reported in the literature.^{[1]}

### 3′-Bromoheptyl-2,3,4,6-O-Acetyl-α-_{D}-Mannopyranoside

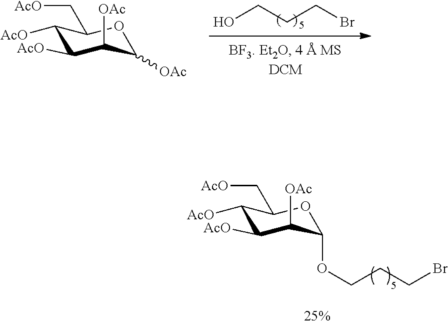

To a solution of mannose pentaacetate (1.76 g, 4.50 mmol) and 3-bromo-1-heptanol (1.32 g, 6.75 mmol) in dry CH₂Cl₂ (4.50 mL) was added BF₃O(Et)₂ (2.3 g, 16.20 mmol) dropwise. After 16 h, the RM was diluted with DCM and washed with NaHCO₃ sat. (10 mL), water (10 mL), dried over MgSO₄, filtered, concentrated in vacuo and purified by column chromatography (20% EtOAc in hexanes 4:1) to give 3′-bromoheptyl-2,3,4,6-O-acetyl-α-_{D}-mannopyranoside (591 mg, 1.12 mmol, 25%) as a pale yellow oil.

¹H NMR 400 MHz, CDCl₃): δ 5.28 (1H, dd, J10.0, 3.4 Hz, H-3), 5.22 (1H, d, J9.9 Hz, H-4), 5.16 (1H, dd, J=3.4, 1.7 Hz, H-2), 4.73 (1H, d, J 1.7 Hz, H-1), 4.21 (1H, dd, J 12.2, 5.3 Hz, H-6a), 4.04 (1H, dd, J 12.2, 2.5 Hz, H-6b), 3.91 (1H, ddd, J 9.8, 5.3, 2.4 Hz, H-5), 3.61 (1H, dt, J 9.6, 6.6 Hz, CH₂—O), 3.44-3.31 (3H, m, CH₂—O CH₂), 2.09 (3H, s, COCH₃), 2.03 (3H, s, COCH₃), 1.98 (3H, s, COCH₃), 1.93 (3H, s, COCH₃), 1.80 (2H, m, CH₂), 1.55 (2H, m, CH₂), 1.39 (2H, m, CH₂), 1.30 (2H, m, 2×CH₂) ppm; ¹³C NMR (101 MHz, CDCl₃): δ 69.1, 66.2, 69.7, 97.5 (C-1), 62.2, 62.5, 68.4, 68.4, 68.4, 68.4, 33.4, 20.92, 20.8, 20.7, 20.7, 32. 7, 28.3, 28.0, 26.00, 28.5 ppm; HRMS(ES): calcd. for C₂₁H₃₃BrO₁₀ 524.13; found C₂₁H₃₃BrO₁₀Na 549.11.

### 3′-Azidoheptyl-2,3,4,6-O-acetyl-α-_{D}-mannopyranoside

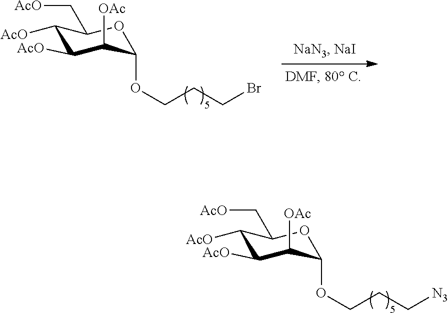

To a solution of 3′-Bromoheptyl-2,3,4,6-O-acetyl-α-_{D}-mannopyranoside (591 mg, 1.12 mmol) in DMF (5 mL) was added sodium azide (132 mg, 2.02 mmol) and sodium iodide (84 mg, 0.56 mmol). The RM was then heated at 80° C. After 16 h the RM was allowed to cool down to room temperature and was then diluted with EtOAc (15 mL), washed with brine (30 mL), the organic phase was dried over MgSO₄, concentrated in vacuo and then purified by column chromatography (25% to 33% EtOAc in hexanes) to give titled compound 3′-azidoheptyl-2,3,4,6-O-acetyl-α-_{D}-mannopyranoside (315 mg, 0.64 mmol, 58%) as a pale yellow oil.

¹H NMR (400 MHz, CDCl₃): δ 5.28 (1H, dd, J 10.0, 3.4 Hz, H-3), 5.21 (1H, m, H-4), 5.16 (1H, dd, J 3.4, 1.8 Hz, H-2), 4.73 (1H, d, J 1.7 Hz, H-1), 4.21 (1H, dd, J 12.2, 5.3 Hz, H-6a), 4.04 (1H, dd, J 12.2, 2.5 Hz, H-6b), 3.91 (1H, ddd, J9.9, 5.3, 2.5 Hz, H-5), 3.61 (1H, dt, J9.7, 6.6 Hz, CH₂—O), 3.38 (1 H, dt, J 9.7, 6.5 Hz, CH₂—O), 3.20 (2H, t, J 6.9 Hz, CH₂), 2.09 (3H, s, COCH₃), 2.03 (3H, s, COCH₃), 1.98 (3H, s, COCH₃), 1.93 (3H, s, COCH₃), 1.59-1.50 (5H, m, 2×CH₂, CHH), 1.31 (5H, dq, J 5.7, 3.1 Hz, 2×CH₂, CHH) ppm; ¹³C NMR (101 MHz, CDCl₃): δ 69.1, 66.3, 69.7, 97.5 (C-1), 62.6, 62.6, 67.6, 68.4, 68.5, 51.4, 20.9, 20.7, 20.7, 20.7, 29.0, 26.1 ppm; HRMS(ES): calcd. for C₂₁H₃₃N₃O₁₀ 487.22; found C₂₁H₃₃N₃O₁₀Na 510.20.

### 3′-O-azidopropyl α-_{D}-mannopyranoside 6

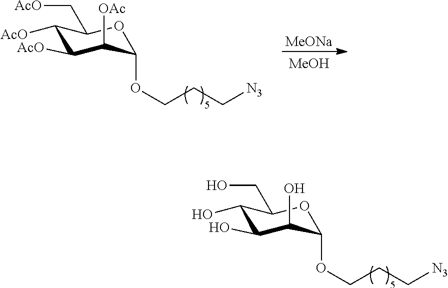

Peracetylated 3′-azidoheptyl-2,3,4,6-O-acetyl-α-_{D}-mannopyranoside (207 mg, 0.65 mmol) was dissolved in MeOH (6.5 mL) then MeONa (4 mg, 0.07 mmol) was added. After 3 hours, the reaction mixture was filtered through a plug of celite and concentrated in vacuo to give3′-azidoheptyl-α-_{D}-mannopyranoside 6 (208 mg, 0.65 mmol, quant.) as a clear oil.

¹H NMR (400 MHz, MeOD): δ 4.64 (1H, d, J 1.6 Hz, H-1), 3.73 (2H, m, H-6a, H-4), 3.66-3.57 (3H, m, H-6b, CHH, H-3), 3.52 (1H, t, J 9.4 Hz, H-2), 3.46-3.39 (1H, m, H-5), 3.32 (1H, dt, J 9.6, 6.2 Hz, CHH), 3.23-3.15 (2H, m, CH₂), 1.55-1.45 (5H, m, 2×CH₂, CHH), 1.37-1.25 (5H, m, 2×CH₂, CHH) ppm; ¹³C NMR (101 MHz, MeOD): δ 100.1, 62.3, 70.9, 61.4, 71.3, 67.1, 67.2, 73.2, 67.1, 48.5, 47.9, 51.0, 29.1, 26.0, 28.6, 29.2 ppm; HRMS(ES): calcd. for C₁₃H₂₅N₃O₆ 319.17; found C₁₃H₂₅N₃O₆Na 342.16.

### 3′-O-aminoheptyl α-_{D}-mannopyranoside 7

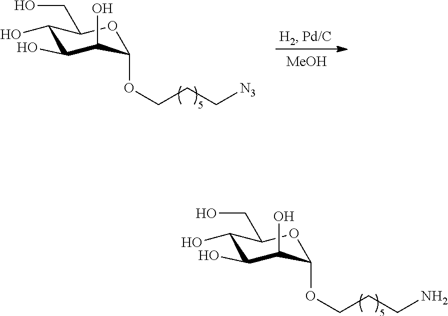

Azide 6 (208 mg, 0.65 mmol) was dissolved in (19:1) EtOH·HCl (2 mL) and the flask was purged three times with nitrogen. Pd/C was then added and the flask was placed under a H₂ atmosphere. After 8 hours, the reaction was filtered through a pad of celite and then concentrated in vacuo. The reaction mixture was purified by HPLC (C18, 5% H₂O in MeCN) to give title compound 3′-aminopropyl α-_{D}-mannopyranoside (76 mg, 0.26 mmol, 40%) as a clear oil. HRMS(ES): calcd. for C₁₃H₂₇NO₆ 293.18; found C₁₃H₂₇NO₆⁺ 294.19.

Glycan Synthesis Specific to K. pneumoniae

### 10′-Bromodecyl-2,3,4,6-O-acetyl-α-_{D}-mannopyranoside

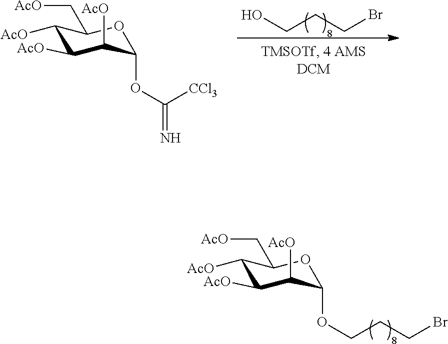

To a flask under N₂, was added the trichloroacetimidate (1 g, 2.0 mmol), and pre-activated 4 molecular sieves (2 g). Dry CH₂Cl₂ (15 mL) and bromodecanol (0.60 mL, 3.0 mmol) were added and the RM stirred for 30 minutes. The reaction was cooled to 0° C. then TMSOTf (0.07 mL, 0.4 mmol) was added dropwise and the reaction stirred at RT for 16 hours. The reaction was quenched with NEt₃ then diluted with CH₂Cl₂ (10 mL), filtered over celite and concentrated in vacuo. Purification by column chromatography (SiO₂, 0-10% acetone in cyclohexane) afforded 10′-bromodecyl-2,3,4,6-O-acetyl-_{D}-mannopyranoside as a white solid (320 mg, 0.56 mmol, 28%).

¹H NMR (400 MHz, CDCl₃): δ 5.35 (1H, dd, J10.0, 3.4 Hz), 5.27 (1H, t, J10.0 Hz), 5.23 (1H, dd, J 3.4, 1.8 Hz), 4.80 (1H, d, J 1.8 Hz), 4.28 (1H, dd, J 12.2, 5.3 Hz), 4.10 (1H, dd, J 12.2, 2.4 Hz), 3.98 (1 H, ddd, J 10.0, 5.3, 2.4 Hz), 3.67 (1 H, dt, J 9.6, 6.8 Hz), 3.48-3.43 (1H, m), 3.41 (2H, t, J 6.9 Hz), 2.16 (3H, s), 2.10 (3H, s), 2.04 (3H, s), 1.99 (3H, s), 1.85 (2H, p, J=7.0 Hz), 1.63-1.55 (2H, m, 2H), 1.46-1.38 (2H, m), 1.35-1.26 (10H, m) ppm.

### 10′-azidodecyl-2,3,4,6-O-acetyl-_{D}-mannopyranoside

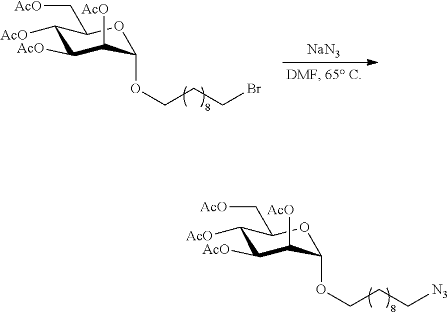

To a solution of 10′-bromodecyl-2,3,4,6-O-acetyl-_{D}-mannopyranoside (300 mg, 0.53 mmol) in dry DMF (5 mL) was added NaN₃ (172 mg, 2.64 mmol) and TBAI (19.5 mg, 0.053 mmol) and the reaction heated to 65° C. overnight. The reaction mixture was concentrated in vacuo then diluted with Et₂O (15 mL) and washed with a saturated aqueous solution of NaHCO₃ (20 mL). The aqueous layer was extracted with Et₂O (15 mL) then the combined organic extracts were washed with brine (2×10 mL). The organic layer was dried with MgSO₄, filtered and concentrated in vacuo, purification by column chromatography (Silica, 5-15% Acetone in cyclohexane) afforded 10′-azidodecyl-2,3,4,6-O-acetyl-_{D}-mannopyranoside as a white solid (150 mg, 0.28 mmol, 53.4%).

¹H NMR (400 MHz, CDCl₃): δ 5.35 (1H, dd, J10.0, 3.5 Hz), 5.27 (1H, t, J10.0 Hz), 5.23 (1H, dd, J 3.5, 1.8 Hz), 4.80 (1H, d, J 1.8 Hz), 4.28 (1H, dd, J 12.3, 5.2 Hz), 4.10 (1H, dd, J 12.3, 2.4 Hz), 3.98 (1H, ddd, J 10.0, 5.2, 2.4 Hz), 3.67 (1H, dt, J9.6, 6.7 Hz), 3.44 (1H, dt, J=9.6, 6.6 Hz), 3.26 (2H, t, J 7.0 Hz), 2.16 (3H, s), 2.10 (3H, s), 2.04 (3H, s), 1.99 (3H, s), 1.64-1.56 (2H, m), 1.37-1.27 (14H, m) ppm.

¹³C NMR (101 MHz, CDCl₃): δ 170.8, 170.3, 170.1, 169.9, 97.7, 77.4, 69.9, 69.3, 68.7, 68.5, 66.4, 62.7, 51.6, 29.5, 29.5, 29.4, 29.3, 29.0, 26.8, 26.2, 20.9, 20.9 ppm.

### 10′-azidodecyl-_{D}-mannopyranoside

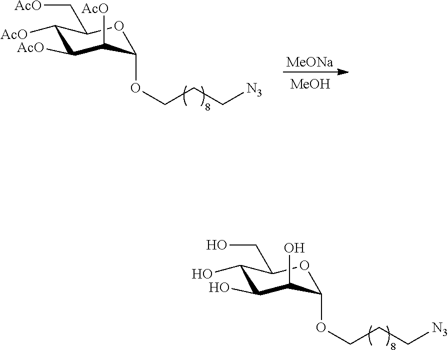

To a solution of 10′-azidodecyl-2,3,4,6-O-acetyl-_{D}-mannopyranoside (126 mg, 0.24 mmol) in MeOH (2.5 mL) was added NaOMe (3.9 mg, 0.072 mmol) and the reaction stirred for 30 minutes until full conversion was observed by TLC. The reaction was neutralised with H⁺ amberlite to pH=6 then filtered and concentrated in vacuo. Column chromatography (Silica, 10% MeOH in CH₂Cl₂) afforded 10′-azidodecyl-D-mannopyranoside as a white solid (42 mg, 0.12 mmol, 48%).

Glycan Synthesis Specific to P. mirabilis

### 2-acetamido-1,3,4,6-O-acetyl-2-deoxy-glucoside

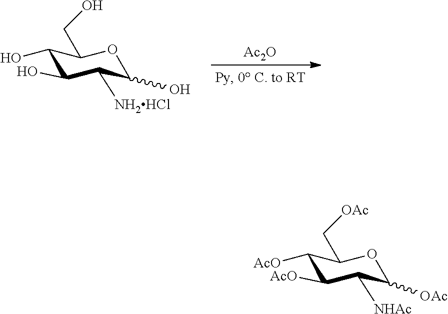

To a solution of _{D}-glucosamine hydrochloride (2 g, 11.1 mmol) in pyridine (5.6 mL) was added Ac₂O (10 mL) at 0° C. After 16 h at RT the RM was concentrated in vacuo and re-dissolved in DCM (20 mL). The organic phase was washed with NaHCO₃ sat. (3×30 mL), HCl 2 M (3×30 mL), H₂O (30 mL), dried over MgSO₄, filtered, concentrated in vacuo and purified by column chromatography (silica, 20 to 60% acetone in cyclohexane) to give 2-acetamido-1,3,4,6-O-acetyl-2-deoxy-glucoside (3.74 g, 9.6 mmol, 86%) as a pale yellow oil.

¹H NMR (400 MHz, CDCl₃): 6.17 (1H, d, J 3.5 Hz), 5.53 (1H, d, J 9.0 Hz), 5.28-5.17 (2H, m), 4.49 (1H, ddt, J 10.4, 6.8, 3.5 Hz), 4.25 (1H, dd, J 12.5, 4.0 Hz), 4.06 (1H, dd, J 12.4, 2.3 Hz), 3.99 (1H, ddd, J9.6, 3.8, 2.3 Hz), 2.20 (3H, s), 2.09 (3H, s), 2.06 (3H, s), 2.05 (3H, s), 1.94 (3H, s) ppm.

### 2-methyl-(3,4,6-tri-O-acetyl-1,2-dideoxy-α-_{D}-glucopyrano)[1,2-d]-oxazoline

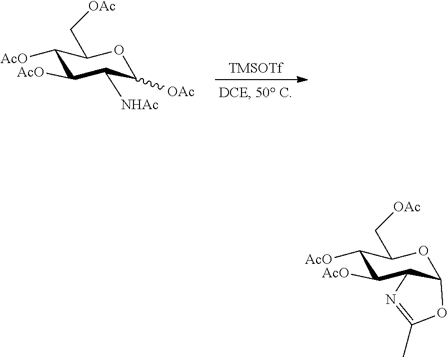

To a solution of 2-acetamido-1,3,4,6-O-acetyl-2-deoxy-glucoside (1 g, 2.57 mmol) in DCE (20 mL) was added TMSOTf (0.5 mL). The RM was then heated at 50° C. for 16 h then neutralised with NEt₃ (1 mL). The organic phase was washed NaHCO₃ sat. (3×30 mL), HCl 2 M (3×30 mL), H₂O (30 mL), dried over MgSO₄, filtered, concentrated in vacuo and purified by column chromatography (silica, 20 to 50% acetone in cyclohexane) to give titled compound 2-methyl-(3,4,6-tri-O-acetyl-1,2-dideoxy-α-_{D}-glucopyrano)[1,2-d]-oxazoline (610 mg, 1.85 mmol, 72%) as a pale yellow oil.

¹H NMR (400 MHz, CDCl₃): δ 6.05 (1 H, d, J 7.5 Hz), 5.27 (1 H, t, J 2.5 Hz), 5.25-5.20 (1H, m), 4.94 (1H, dt, J 9.2, 1.7 Hz), 4.19 (1H, s), 3.63 (1H, dt, J 8.8, 4.4 Hz), 3.14 (1H, qd, J 7.3, 4.9 Hz), 2.11 (3H, s), 2.10 (3H, s), 2.09 (3H, s) ppm.

### 2-acetamido-3′-chloropropyl-2-deoxy-β-D-glucopyranoside

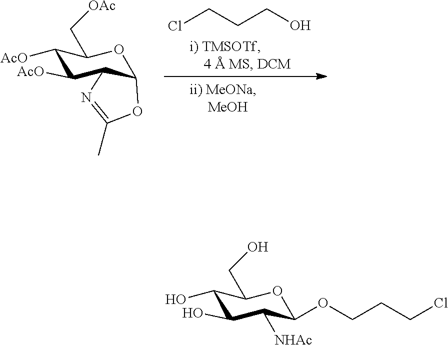

To a solution of 2-methyl-(3,4,6-tri-O-acetyl-1,2-dideoxy-α-_{D}-glucopyrano)[1,2-d]-oxazoline (610 mg, 1.85 mmol) in dry DCM (6 mL) was added pre-activated 4 A MS (427 mg) followed by chloropropanol (0.23 mL, 2.75 mmol). After 1 h TMSOTf (0.17 mL, 1.0 mmol) was added. After 16 h the RM was neutralised with Et₃N (1 mL) and filtered through celite (filter cake washed with DCM/MeOH 9/1, 20 mL). The filtrate was then concentrated in vacuo and re-dissolved in DCM (20 mL) and NaHCO₃ sat. solution (20 mL), the organic phase was separated and washed with NaHCO₃ sat. solution (2×20 mL), 2 M HCl (2×20 mL), H₂O (20 mL), dried over MgSO₄, filtered, and concentrated in vacuo. The crude residue was dissolved in dry MeOH (15 mL) MeONa (49 mg, 0.9 mmol) was then added. After 2 h the RM was not finished thus additional MeONa (49 mg, 0.9 mmol) was added then the reaction placed at −4° C. for 16 h. The RM was then neutralised with amberlite H⁺ to pH=7, filtered, concentrated in vacuo and purified by column chromatography (silica, 5 to 20% MeOH in DCM) to 2-acetamido-3′-chloropropyl-2-deoxy-pi-D-glucopyranoside (205 mg, 0.69 mmol, 37% over 2 steps) as an off-white solid.

¹H NMR (400 MHz, D₂O): δ 4.50 (1H, d, J 8.4 Hz, H-1), 4.01 (1H, dt, J 10.3, 5.3 Hz), 3.91 (1H, dd, J 12.3, 1.7 Hz), 3.78-3.59 (6H, m), 3.56-3.47 (1H, m), 3.45-3.42 (2H, m), 2.04 (3H, s), 2.03-1.93 (2H, m) ppm.

### 2-acetamido-3′-azidopropyl-2-deoxy-β-_{D}-glucopyranoside

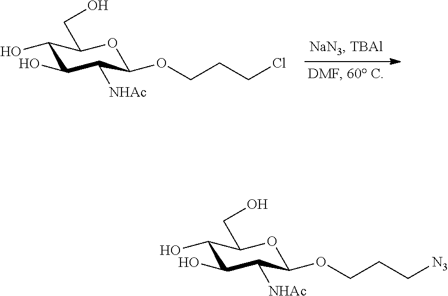

To a solution of 2-acetamido-3′-chloropropyl-2-deoxy-pi-d-glucopyranoside (196 mg, 0.66 mmol) in DMF (5 mL) was added sodium azide (248 mg, 3.81 mmol) followed by TBAI (24 mg, 0.06 mmol). The RM was then placed at 60° C. After 16 h the RM was concentrated in vacuo then re-dissolved in DCM (10 mL) and H₂O (10 mL). The aqueous phase was extracted with DCM (3×10 mL), the combined organic phases were then washed with NaHCO₃ sat. solution (2×10 mL), 2 M HCl (2×10 mL), H₂O (10 mL), dried over MgSO₄, filtered, concentrated in vacuo and then purified by column chromatography (silica, 10% MeOH in DCM) to give 2-acetamido-3′-azidopropyl-2-deoxy-β-_{D}-glucopyranoside (155 mg, 0.51 mmol, 74%) as an off-white solid.

¹H NMR (400 MHz, D₂O): δ 4.49 (1H, d, J 8.3 Hz, H-1), 3.97 (1H, dt, J 10.6, 5.4, 5.4 Hz), 3.91 (dd, J 12.4, 1.7 Hz), 3.77-3.60 (4H, m), 3.57-3.49 (1H, m), 3.46-3.41 (2H, m), 3.36 (2H, td, J 6.7, 1.7 Hz), 2.03 (3H, s), 1.83 (2H, quint, J 6.3 Hz, 2H) ppm; ¹³C NMR (101 MHz, D₂O): δ 174.6, 101.2, 75.9, 73.8, 70.0, 67.2, 60.8, 55.6, 47.8, 28.1, 22.2 ppm.

### 2-acetamido-3′-aminopropyl-2-deoxy-β-_{D}-glucopyranoside

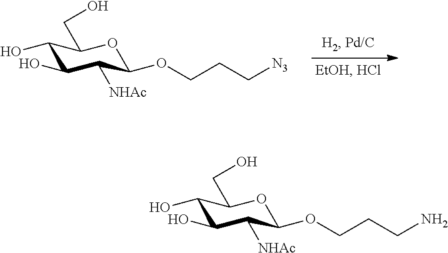

To a solution of 2-acetamido-3′-azidopropyl-2-deoxy-β-d-glucopyranoside (141 mg, mmol) in EtOH (3 mL) under N₂ atm. was added Pd/C (3 mg). Conc. HCl (0.04 mL, mmol) was then added. The RM was then subjected to 3 cycles vacuum/H₂ and then placed under a H₂ atm. After 16 h the RM was filtered through celite, (filter cake washed with 7 N NH₃ in MeOH 20 mL) and concentrated in vacuo to give desired product 2-acetamido-3′-aminopropyl-2-deoxy-β-d-glucopyranoside (90 mg, 0.32 mmol, 63%) as a pale brown oil.

¹H NMR (400 MHz, D₂O): δ (selected peaks) 4.47 (1 H, d, J 8.4 Hz H-1), 3.06 (2H, t, J 7.1 Hz), 2.03 (3H, s), 1.93 (2H, qint, J 6.2 Hz) ppm.

Glycan Synthesis Specific to P. aeruginosa

### 3′-chloropropyl-fucopyranoside

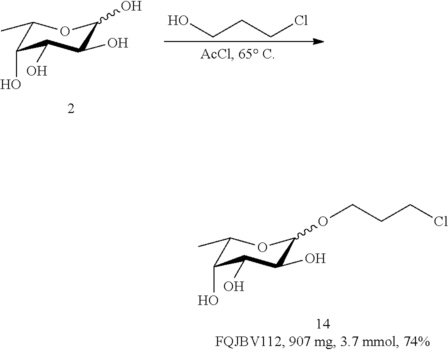

To a solution of _{L}-fucose(821 mg, 5 mmol) in chloropropanol (4.2 mL, 50 mmol) was added acetyl chloride (0.71 mL, 10 mmol) dropwise. The RM was then heated at 65° C. for 4 h. After allowing the RM to reach RT the RM was neutralised with NaHCO₃ (solid) until pH=7 then concentrated in vacuo and purified by chromatography (5% to 10% MeOH in DCM) to give titled compound 3′-chloropropyl-fucopyranoside as a pale yellow oil (907 mg, 3.7 mmol, 74%).

¹H-NMR (400 MHz, D₂O) δ (ratio α/β): 4.87 (1 H, d, J 4.0 Hz), 4.36 (0.35 H, d, J 8.0 Hz), 4.08 (1 H, q, J 6.6 Hz), 4.00 (0.35 H, q, J 6.6 Hz), 3.91-3.54 (9H, m), 3.44 (0.35 H, dd, J 10.0, 7.9 Hz), 2.07 (3H, dtd, J 17.6, 14.6, 13.3, 7.4 Hz, 3H), 1.24 (1H, d, J 6.5 Hz), 1.20 (3H, d, J 6.6 Hz) ppm.

### 3′-azidopropyl-fucopyranoside

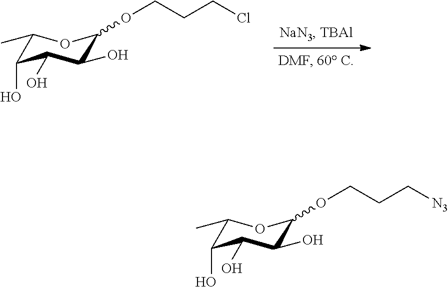

To a solution of 3′-chloropropyl-fucopyranoside (241 mg, 1 mmol) in DMF (8 mL) was added NaN₃ (376 mg, 5.78 mmol) and TBAI (46 mg, 0.12 mmol). The RM was then heated at 60° C. for 16 h then concentrated in vacuo. The crude residue was then purified by column chromatography (10% MeOH in DCM) to give titled compound 3′-azidopropyl-fucopyranoside as a pale yellow oil (249 mg, 1 mmol, quant., α/β: 2/1).

¹H-NMR (400 MHz, D₂O) δ (ratio α/β): 4.86 (1 H, d, J 3.7 Hz), 4.21 (1 H, d, J 6.8 Hz), 4.00-3.89 (2H, m), 3.86-3.50 (8H, m), 3.42 (3H, qd, J 7.0, 6.6, 2.3 Hz), 1.99-1.82 (3H, m), 1.33 (1 H, d, J 6.6 Hz), 1.29 (3H, d, J 6.7 Hz) ppm.

Alternative probes using different sugars were synthesised as described in Carb. Res. 1995, 266, 211-219 and Org. Biomol. Chem., 2018, 16, 74307437.

Glycan Synthesis with m-(α-Azidoacetamido)-Phenyl

### m-nitrophenyl 2,3,4,6-tetra-O-acetyl-_{D}-galactoside

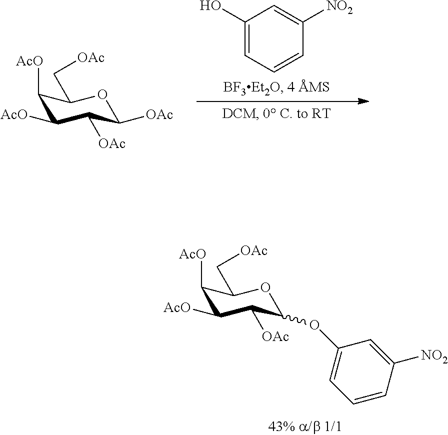

Following a protocol from Titz and coworkers. To a solution of peracetylated galactose (1.56 g, 3.99 mmol) and m-nitrophenol (686 mg, 4.93 mmol) in dry DCM (8 mL) was added preactivated 4 Å MS (1.77 g). After 30 min BF₃·Et₂O (2.56 mL, 20.7 mmol) was added. After 16 h the RM was neutralised with NEt₃ (5 mL), filtered through celite, the filter cake was washed with DCM/MeOH solution (20 mL). The filtrate was then washed with NaHCO₃ aq. solution (20 mL), 2 M HCl (2×20 mL), H₂O (20 mL), dried over MgSO₄, filtered, concentrated and then purified by column chromatography (silica, 10 to 20% acetone in cyclohexane) to give a mixture of desired nitrophenyl galactoside (800 mg, 1.70 mmol, 43%, ratio α/β: 1/1) as a pale yellow solid.

¹H NMR (400 MHz, CDCl₃) δ (ratio α/β1/1): 7.97-7.92 (2H, m), 7.89 (3H, dt, J6.3, 2.0 Hz), 7.46 (3H, td, J 8.1, 1.5 Hz), 7.37-7.29 (2H, m), 5.41 (1 H, dt, J 6.9, 4.3 Hz), 5.35-5.32 (4H, m), 5.13 (2H, d, J 8.2 Hz), 4.39 (1H, dd, J 5.4, 4.3 Hz), 4.22-4.13 (4H, m), 4.09 (2H, dd, J 6.8, 3.7 Hz), 2.18 (3H, s), 2.14 (3H, s), 2.09 (3H, s), 2.08 (3H, s), 2.03 (3H, s), 2.01 (6H, s), 2.00 (3H, s), 1.99 (3H, s) ppm.

### m-(α-Azidoacetamido)-phenyl 2,3,4,6-tetra-O-acetyl-_{D}-galactopyranoside

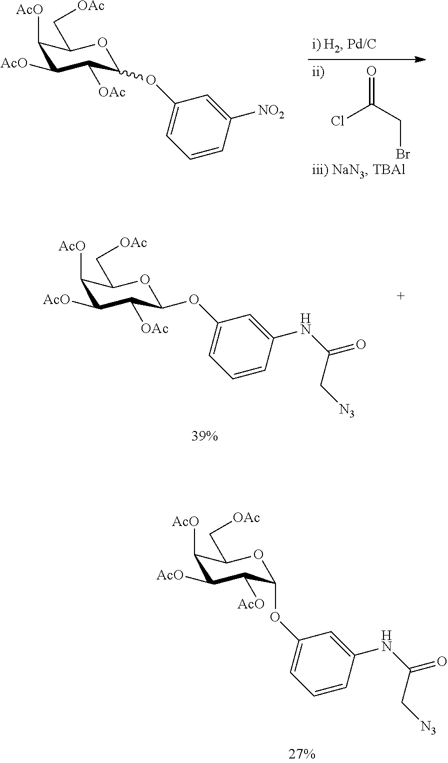

To a solution of m-nitrophenyl 2,3,4,6-tetra-O-acetyl-_{D}-galactoside (742 mg, 1.58 mmol) in DCM (35 mL) was added Pd/C (10% loading, 74 mg). The RM was then subjected to three cycles Vacuum/H₂ and then placed under an atmosphere of H₂ for 18 h. The RM was then cooled at 0° C. and NEt₃ (260 μL, 1.89 mmol) was added followed by the dropwise addition of bromo-acetylbromide (159 μL, 1.91 mmol). After an additional hour at 0° C. the RM was allowed to warm to RT and stirred for an additional hour. The reaction was then filtered through celite, the filtrate was then washed with NaHCO₃ aq solution (3×20 mL), 2M HCl, H₂O (2×20 mL), dried over MgSO₄, filtered and concentrated in vacuo. The crude residue was dissolved in DMF (20 mL) then NaN₃ (576 mg, 8.86 mmol) was added followed with TBAI (60 mg, 0.16 mmol) then the RM was heated at 50° C. After 16 h the RM was concentrated in vacuo and purified by column chromatography (silica, 10 to 40% acetone in cyclohexane) to afford first the α-glycoside (343 mg, 0.66 mmol, 39%) and then the p-glycoside (242 mg, 0.46 mmol, 27%) both as pale yellow oils.

### m-(α-Azidoacetamido)-phenyl 2,3,4,6-tetra-O-acetyl-α-_{D}-galactopyranoside

¹H NMR (400 MHz, CDCl₃) δ:

8.03 (1H, d, J 5.6 Hz), 7.36 (1H, t, J 2.2 Hz), 7.25 (2H, t, J 8.0 Hz), 7.17 (1H, ddd, J 8.0, 2.1, 1.0 Hz), 6.83 (1H, ddd, J 8.2, 2.4, 1.0 Hz), 5.42 (1H, ddd, J 7.0, 4.6, 3.6 Hz), 5.32 (1H, dd, J 2.0, 0.7 Hz), 5.13-5.09 (1H, m), 4.39 (1 H, dd, J 5.7, 3.7 Hz), 4.35 (1 H, dd, J 11.9, 4.8 Hz), 4.20 (1 H, dd, J 11.9, 7.1 Hz), 4.14 (2H, s), 4.13-4.03 (1H, m), 2.15 (3H, s), 2.14 (3H, s), 2.13 (3H, s), 1.99 (3H, s) ppm.

### m-(α-Azidoacetamido)-phenyl 2,3,4,6-tetra-O-acetyl-β-_{D}-galactopyranoside

¹H NMR (400 MHz, CDCl₃) δ: 8.01 (1H, br. s), 7.41 (1H, t, J 2.2 Hz), 7.26 (3H, t, J 8.3 Hz), 7.16 (1 H, ddd, J 8.1, 2.1, 1.0 Hz), 6.80 (1 H, ddd, J 8.2, 2.4, 1.0 Hz), 5.48 (1 H, dd, J 10.4, 7.9 Hz), 5.46 (1H, dd, J=2.9, 0.9 Hz), 5.11 (1 H, dd, J 10.4, 3.5 Hz), 5.07 (1 H, d, J 8.0 Hz), 4.23 (1 H, dd, J 11.3, 6.9 Hz), 4.17 (1H, dd, J 11.3, 6.2 Hz), 4.15 (2H, s), 4.08 (1H, td, J 6.6, 1.2 Hz), 2.18 (3H, s), 2.08 (3H, s), 2.06 (3H, s), 2.01 (3H, s) ppm.

### m-(α-Azidoacetamido)-phenyl β-_{D}-galactopyranoside

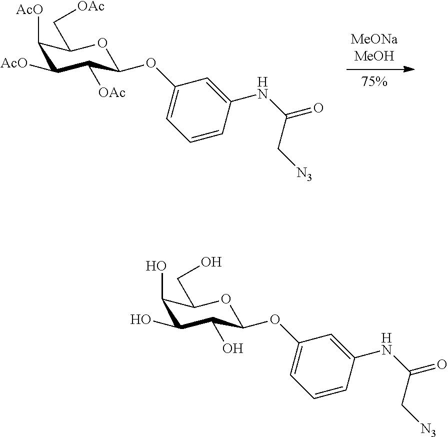

To a solution of galactose m-(α-Azidoacetamido)-phenyl 2,3,4,6-tetra-O-acetyl-β-_{D}-galactopyranoside (77 mg, 0.15 mmol) in MeOH (1 mL) was added MeONa (6 mg, 0.11 mmol). After 2 h the RM was neutralised using amberlist H⁺ to pH=7 then filtered, concentrated in vacuo and purified by column chromatography (silica, MeOH in DCM) to give the titled galactoside m-(α-Azidoacetamido)-phenyl β-_{D}-galactopyranoside (39 mg, 0.11 mmol, 75%) as an off-white solid.

¹H NMR (400 MHz, CD₃OD) δ: 7.44 (2H, t, J2.0 Hz), 7.26-7.19 (2H, m), 6.91-6.86 (1H, m), 4.87 (1H, d, J 7.8 Hz), 4.00 (s, 2H), 3.91 (1H, dd, J 3.4, 1.0 Hz), 3.82-3.73 (3H, m), 3.72-3.68 (1H, m), 3.58 (1H, dd, J9.7, 3.4 Hz) ppm.

### m-(α-Azidoacetamido)-phenyl α-_{D}-galactopyranoside

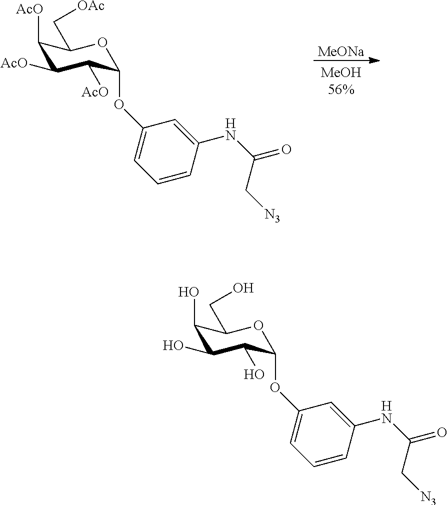

To a solution of peracetylated galactose m-(α-Azidoacetamido)-phenyl β-_{D}-galactopyranoside (77 mg, 0.15 mmol) in MeOH (1 mL) was added MeONa (6 mg, 0.11 mmol). After 2 h the RM was neutralised using amberlist H⁺ to pH=7 then filtered, concentrated in vacuo and purified by column chromatography (silica, 10% MeOH in DCM) to give the titled galactoside m-(α-Azidoacetamido)-phenyl α-_{D}-galactopyranoside (29 mg, 0.08 mmol, 56%) as an off-white solid.

¹H NMR (400 MHz, CD₃OD) δ: 7.38 (1H, t, J 2.2 Hz), 7.24-7.20 (1H, m), 7.20-7.14 (1H, m), 6.87-6.81 (1H, m), 5.51 (1 H, d, J 2.2 Hz), 4.23 (1 H, dd, J 4.2, 2.3 Hz), 4.15 (1 H, dd, J 6.7, 4.3 Hz), 4.08 (1 H, dd, J 6.7, 3.0 Hz), 4.00 (2H, s), 3.80-3.72 (1H, m), 3.65-3.60 (2H, m).ppm.

### 1-amino-_{L}-fucose

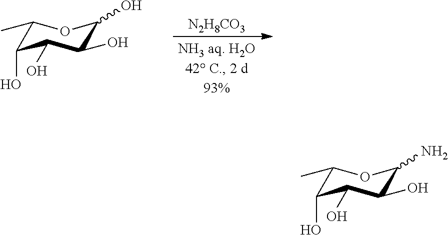

Adapting a procedure from Lubineau and co-workers (Lubineau, J. Augé, B. Drouillat, Car. Res. 1995, 266, 211-219.). To a solution of _{L}-fucose (511 mg, 3.11 mmol) in H₂O (14 mL) was added N₂H₈CO₃ (326 mg, 3.39 mmol) followed by a solution of aqueous ammonia (35% in H₂O, 15.6 mL). The RM was then heated at 42° C. for 2 d then concentrated in vacuo to give 1-amino-L-fucose (470 mg, 2.88 mmol, 93%) as a pale yellow solid. Data consistent with the literature (L. M. Likhosherstov, O. S. Novikova, V. N. Shibaev, Doklady Chem. 2003, 389, 73-76).

¹H-NMR (400 MHz, D₂O) δ: 4.00 (1 H, d, J 8.7 Hz,), 3.77-3.72 (3H, m), 3.61 (1 H, dd, J 9.8, 3.4 Hz), 3.33 (1H, dd, J9.6, 8.7 Hz), 1.20 (3H, d, J 6.5 Hz) ppm.

### 1-NH₂-6-O-(α-_{D}-Galactopyranosyl)-_{D}-glucopyranose

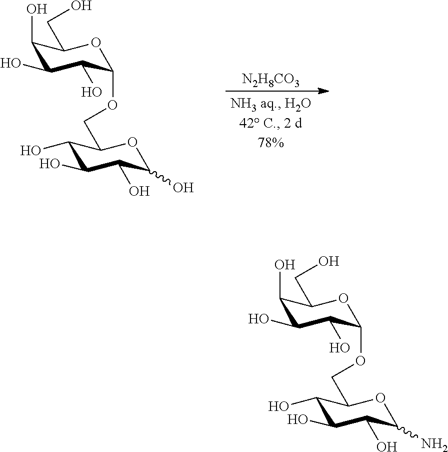

To a solution of melibioze (50 mg, 0.15 mmol) in NH₃ aq. solution (35%, 0.76 mL) was added NH₂H₈CO₃ (15 mg, 0.16 mmol) in H₂O (0.76 mL). The RM was heated at 42° C. for 2 d then concentrated in vacuo to give the 1-NH2-6-O-(α-_{D}-Galactopyranosyl)-_{D}-glucopyranose (39 mg, 0.11 mmol, 78%) as a pale orange foam.

¹H NMR (400 MHz, D₂O) δ (ratio α/β): 4.96 (2H, d, J 3.8 Hz), 4.11 (1 H, d, J 8.8 Hz,), 3.97 (3H, d, J 3.7 Hz), 3.95-3.77 (5H, m), 3.72 (3H, d, J 6.5 Hz), 3.48-3.44 (2H, m), 3.20-3.12 (1H, m) ppm·1NH₂—N-acetyl-D-lactosamine

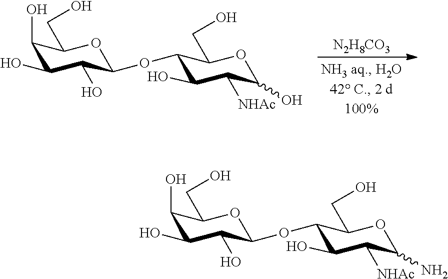

To a solution of N-acetyl-D-lactosamine (20 mg, 0.05 mmol) in NH₃ aq. solution (35%, 0.26 mL) was added NH₂H₈CO₃ (5 mg, 0.05 mmol) in H₂O (0.26 mL). The RM was heated at 42° C. for 2 d then concentrated in vacuo to give the 1 NH₂—N-acetyl-D-lactosamine (20 mg, 0.05 mmol, 100%) as a pale orange foam.

¹H NMR (400 MHz, D₂O) δ (ratio α/β: 3/1): 4.47-4.38 (2H, m), 4.00-3.83 (4H, m), 3.78-3.61 (7H, m), 3.55-3.45 (2H, m), 2.06 (1 H, s), 2.01 (3H, s) ppm.

Sugars with specificity towards a variety of microbial pathogens are listed in Table 2.

TABLE 2

| | | | | | | |
|---|---|---|---|---|---|---|
| 1 | K. pneumoniae | GalNAc-β-(1- | L-sorbose | Ribitol | Man | GalNAc-β-(1- |
| | | 4)-Gal | (ferment) | (ferment) | | 4)-Gal-β-(1- |
| | | | | | | 4)-Glc |
| 2 | P. mirabilis | Xylose | GalNAc-β- | β-Gal | Lactose | Galactose |
| | | | (1-4)-Gal | | | |
| 3 | S. saprophyticus | Gal-β-(1-4)- | | | | |
| | | GlcNAc | | | | |
| 4 | C. albicans | GalNAc-β-(1- | Gal-β-(1-3)- | Gal-β-(1-4)- | | |
| | | 4)-Gal | GalNAc | Glc | | |
| 5 | C. trachomatis | GalNAc-β-(1- | Gal-β-(1-3)- | GalNAc-β- | | |
| | | 4)-Gal | GalNAc | (1-4)-Gal-β- | | |
| | | | | (1-4)-Glc | | |
| 6 | N. gonorrhoeae | GlcNAc-β-(1- | Gal-β-(1-4)- | Gal-α-(1-3)- | Gal-α-(1- | β-Gal |
| | | 3)-Gal-β-(1-4)- | Glc | Gal | 4)-Gal | |
| | | Glc | | | | |
| 7 | Treponema | Glc | | | | |
| | pallidum | | | | | |
| 8 | P aeruginosa | Gal | fuc | Fuc-α-(1- | Gal-α-(1- | |
| | | | | 2)-Gal | 6)-Glc | |
| 9 | E. coli | Man | | | | |
| 10 | E. cloacae | Man | Gal | Lactose | Gal-α-(1- | Neu5Ac |
| | | | | | 6)-Glc | |
| 11 | E. Faecalis | Fuc | Gal | | | |
| 12 | S. epidermidis | GlcNAc-β-(1- | Heparin | | | |
| | | 6)-GlcNAc | sulfate | | | |
| | | | (various | | | |
| | | | patterns) | | | |
| 13 | S. aureus | Neu5Ac | Neu5Gc | sLex | aGM1 | Neu5Ac-α- |
| | | | | | | (2-3)-Gal |
| 14 | S. pyogenes | Neu5Ac-α-2-6- | | | | |
| | | GalNAc | | | | |
| 15 | S. agalactiae | sLex | sLeA | LeY | LeB | |

Any linker described in the above experimental and embodiments of the application can be used with probes 1 to 15 as described in Table 2.

### Spacer Synthesis

The synthesis of the spacer if applicable started with the mono-tosylation of tetraethylene glycol which afforded cleanly the desired tosylate in excellent yield. The tosylate was then displaced using sodium azide in DMF giving desired azide in moderate yield. The reduction was then performed following a procedure described by Heller in 2015.

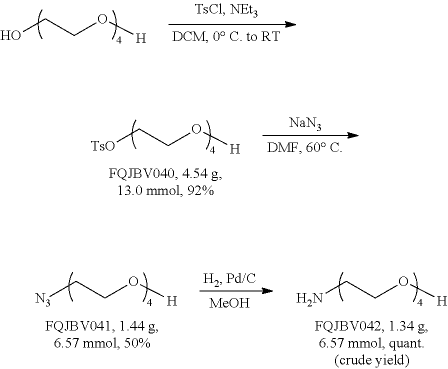

### Tetraethylene glycol p-toluenesulfonic acid ester

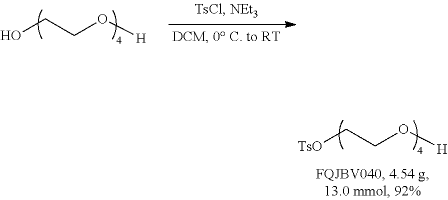

To a solution of tosyl chloride (2.68 g, 14.1 mmol) in DCM (27 mL) was added tetraethylene glycol (24.3 mL, 141 mmol). The RM was then cooled to 0° C. then NEt₃ (2.95 mL,) was added dropwise. The RM was then allowed to reach RT and left to stir overnight. After 16 h the RM was diluted with DCM (30 mL) and H₂O (50 mL). The aqueous phase was separated and extracted with DCM (3×30 mL). The combined organic phases were washed with H₂O (2×30 mL), 2 M HCl (2×30 mL), NaHCO₃ sat. solution (2×30 mL), H₂O (30 mL), dried over MgSO₄, filtered and concentrated in vacuo to give titled compound as a colourless oil (4.54 g, 13.0 mmol, 92%). The product was used in the next step without further purification. Data are in accordance with the literature.

¹H-NMR (400 MHz, CDCl₃) δ: 7.79 (2H, d, J 8.1 Hz, ArH), 7.34 (2H, d, J 8.3 Hz, ArH), 4.15 (2H dd, J 5.4, 4.2 Hz, CH₂—OTs), 3.72-3.57 (8H, m, CH₂), 3.59 (3H, s, CH₃) ppm

### 2-(2-(2-(2-azidoethoxy)ethoxy)ethoxy)ethanol

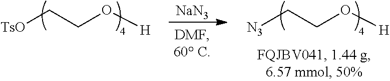

To a solution of tosylate (4.94 g, 13.0 mmol) in DMF (50 mL) was added NaN₃ (1.02 g, 15.7 mmol). The RM was heated at 60° C. for 16 h then concentrated under high vacuum. The crude residue was diluted with EtOAc (50 mL) and H₂O (50 mL). The aqueous phase was separated and extracted with EtOAc (3×50 mL). The combined organic phases were washed with brine (4×50 mL), dried over MgSO₄, filtered, concentrated and then purified by column chromatography (30 to 50% acetone in cyclohexane) to give desired azide as a colourless oil (1.44 g, 6.57 mmol, 50%). Data in accordance with the literature.

¹H-NMR (400 MHz, CDCl₃) δ: 3.75-3.71 (2H, m, CH₂), 3.70-3.65 (CH₂, m, 10H), 3.63-3.60 (2H, m, CH₂), 3.43 (2H, t, 3.4 Hz, m, CH₂N₃) ppm.

### 2-(2-(2-(2-aminoethoxy)ethoxy)ethoxy)ethanol

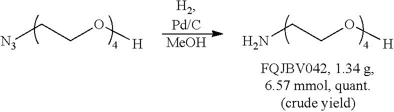

To a solution of azide (1.44 g, 6.57 mmol) in MeOH (12 mL) under a nitrogen atmosphere was added Pd on charcoal (10% w/w, 144 mg,). The RM was then subjected to 3 cycles of H₂/vacuum then placed under H₂ atmosphere. After 16 h the RM was filtered through celite, the filter cake was then washed with MeOH (20 mL). The filtrate was then concentrated. Some starting material remained thus another hydrogenation was set up as above. After another 16 h the RM was filtered through celite, the filter cake washed with MeOH (20 mL) and the filtrate concentrated to give the desired amine as a colourless oil (1.34 g, 6.57 mmol, quant.). The product was reacted in the next step without further purification as the NMR was clean from any other product. Data in accordance with the literature.

¹H-NMR (400 MHz, CDCl₃) δ: 3.75-3.70 (2H, m, CH₂), 3.63 (s, 10H, 5×Ch₂), 3.61-3.56 (2H, m, CH₂), 2.91 (t, J 5.0 Hz, 6H).

A number of spacers are described in Stephen A. Hill et al. Nanoscale, 2016, 8, 18630-18634 and be used in the above-described composition.

### Example 2—Pathogen Detection

The pathogen Proteus Mirabilis was detected in liquid samples by agglutination. The probe used was a 1-amino xylose complex attached to 10 μm latex beads.

### Materials

50 μl probe (functionalised beads in PBS)
Phosphor Buffer Saline (PBS)
10⁷ P. Mirabilis in TSB
Blank Tryptic Soy Broth (TSB)
Microscope
Eppendorf
LP Vortex Mixer
IKS Mixer Lab Dancer Vortex

### Method

Culture
1. Set up an overnight culture of P. Mirabilis in TSB on a shaker incubator (37° C. at 200 rpm).
2. Dilute the overnight culture of P. Mirabilis to a concentration of 10⁷ cfu/ml in TSB.

Sample Preparation
3. Pipette 20 μl P. Mirabilis sample into Eppendorf.
4. Then add 10 μl of probe and solution for even distribution of beads. This can be done by vortex or by hand.
54. Vortex mix the probe and bacteria sample briefly.

Incubation at Room Temperature (22° C.)
6. Vortex mix for 15 minutes (1500 rpm with LP vortex mixer in continuous mode).
7. Rest for 10 mins.

Data Collection
8. Pipette sample unto standard microscope slide
9. Image with microscope to detect agglutination

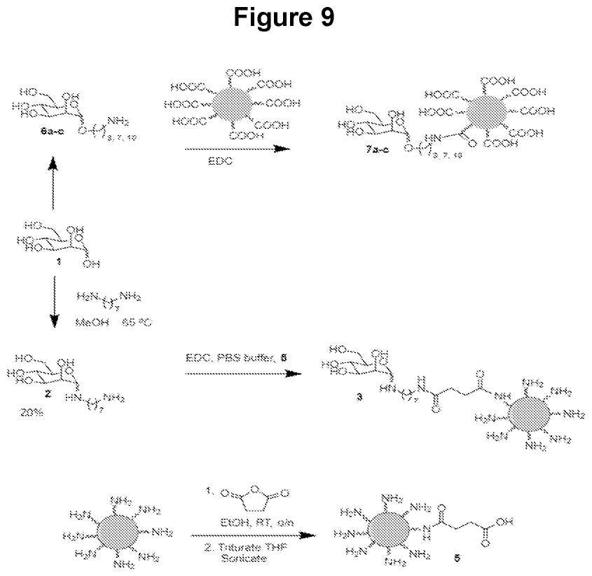

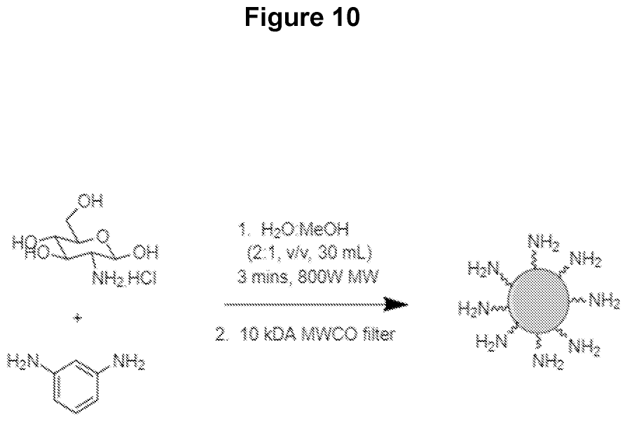

### Example 3—Probe Synthesis of Mannoside Probes (See Also FIG. 9 to 14)

A series of mannoside probes were synthesised according to the general schemes set out in FIG. 9. Route A and Route B shown in FIG. 9 both start with mannose 1 and convert it to an aminoalkyl glycoside 2 and 6a-c. Both then utilise the coupling of an aminoalkyl glycoside to a particle. In Route A, the aminoalkyl glycoside 6a-c is coupled directly to a particle having surface carboxylate groups. In Route B, the particle has surface amines and requires a linker (see insert) to facilitate the coupling. The linker is succinate, which changes the functional group available for binding from an amine to a carboxylic acid (for clarity, only one succinate group is shown although the particle will of course be bound by many more succinate groups). Coupling to compound 2 is then done using standard amide coupling conditions, such as EDC, to give probe 3. In Route A the glycosidic atom is oxygen and in Route B the glycosidic atom is nitrogen.

The synthesis of certain specific compounds is detailed below.

### ((2S,3S,4S,5S,6R)-2-((7-aminoheptyl)oxy)-6-(hydroxymethyl)tetrahydro-2H-pyran-3,4,5-triol)

### Compound 2

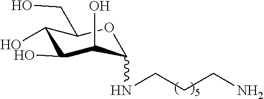

1,7-Diaminoheptane (0.5 g, 3.84 mmol) and mannose (0.69 g, 3.84 mmol) in 5 mL of methanol was stirred at 60° C. for 2 hours. Reduce under vacuum before being purified with reverse-phase preparative HPLC (5% to 95% MeOH w/0.05% formic acid in H₂O w/0.05% formic acid) to give the product 2 as a slightly yellow oil (60 mg, 5%). ¹HNMR (CDCl₃, 400 MHz): δ 1.29 (s, 6H, c); 1.56 (m, 2H, d); 1.62 (m, 2H, b); 2.89 (t, J=7.6 Hz, 2H, a); 3.01 (t, J=6.85 Hz, 2H, e); 3.42-4.10 (m, 7H, sugar protons including anomeric). ¹³C-NMR (CDCl₃, 101 MHz): δ 25.8, 26.3, 28.1, 28.5, 30.5, 40.2, 44.5, 61.1, 67.2, 71.1, 73.8, 77.2, 86.5. MS-ESI of C₁₃H₂₈N₂O₅ calc: 292.20 found 293.21 [M+H*].

Control compounds were made using the same procedure, except that lactose was used in the place of mannose.

### Compound 4

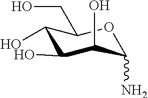

1-amino mannose was prepared using the microwave assisted Kochetkov amination protocol of the corresponding unprotected glycosides with ammonium carbonate (5-fold excess w/w over sugar). (Bejugam, M.; Flitsch, S. L. Org Lett 2004, 6, 4001).

### Compound 6a; 3—Mannoside

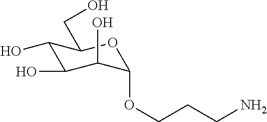 [SMILES: NCCCOC1OC(CO)C(O)C(O)C1O]

¹H NMR (400 MHz, deuterium oxide) δ 4.82 (d, J=2.2 Hz, 1H), 3.92 (dt, J=3.7, 1.9 Hz, 1H), 3.87 (d, J=1.7 Hz, OH), 3.83 (t, J=2.4 Hz, 1H), 3.75 (dtd, J=22.3, 11.9, 5.8 Hz, 1H), 3.64-3.48 (m, 2H), 3.08 (tt, J=9.8, 4.8 Hz, 1H), 2.01-1.88 (m, 1H). ¹³C NMR (101 MHz, D₂O) δ 99.7, 70.0, 61.0, 61.0, 70.5, 60.97, 66.76, 72.82, 64.97, 37.52, 26.62. HRMS(ES): calculated for C₉H₁₉NO₆ 237.12; found C₉H₁₉NO₆⁺ 238.12.

### Compound 6b: 7—Mannoside

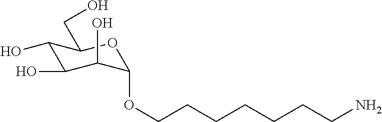 [SMILES: NCCCCCCCOC1OC(CO)C(O)C(O)C1O]

¹H NMR (400 MHz, D₂O) δ 4.88 (d, J=1.7 Hz, 1H, H-1), 3.95 (dd, J=3.5, 1.8 Hz, 1H, H-2), 3.90 (dd, J=12.2, 1.9 Hz, 1H, H-6a), 3.84-3.72 (M, 3H, H-3, H-6b, OCHH), 3.72-3.63 (M, 2H, H-4, H-5), 3.57 (dtd, J=8.2, 5.7, 5.1, 2.5 Hz, 1H, OCHH), 2.74-2.68 (M, 2h, CH₂NH₂), 1.76-1.30 (M, 10h, CH₂). ¹³C NMR (100 MHz, D₂O) δ 99.9 (C-1), 72.8 (C-5), 70.5 (C-3), 70.1 (C-2), 67.9 (OCH₂), 66.9 (C-4), 60.9 (C-6), 48.3 (CH₂NH₂), 28.4 (CH₂), 28.2 (CH₂), 27.5 (CH₂), 26.2 (CH₂), 25.3 (CH₂). HRMS(ES): calculated for C₁₃H₂₇NO₆ 293.18; found C₁₃H₂₇NO₆⁺ 294.19.

### Compound 6c; 10—Mannoside

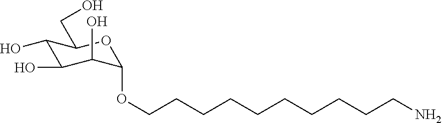 [SMILES: NCCCCCCCCCCOC1OC(CO)C(O)C(O)C1O]

¹H NMR (400 MHz, D₂O) δ 7.41 (s, 2H, NH₂), 4.80 (d, J<2 Hz, 1H, H-1), 3.90-3.78 (m, 2H, H-3, H-6a), 3.75-3.53 (m, 5H, H-4, H-6b, —CHH—, H-5, H-2), 3.52-3.44 (m, 1H, —CHH—), 2.93 (t, J=7.6 Hz, 2H, —CH₂—), 1.57 (tq, J=12.8, 7.4 Hz, 4H, 2×—CH₂—), 1.37-1.20 (m, 12H, 6×—CH₂—). ¹³C NMR (101 MHz, D₂O) δ 99.6 (C1), 72.70, 70.7, 70.1, 67.9, 66.7, 60.9, 39.6, 28.5, 28.4, 28.4, 28.1, 26.7, 25.5, 25.3. HRMS(ES): calculated for C₁₆H₃₃NO₆ 335.23; found C₁₆H₃₃NO₆⁺ 336.23.

General Procedure for Functionalisation of Particles with Aminated Carbohydrates.

Acid functionalised soluble particles were added to a PBS buffer solution (ph 7.4, 3 ml) and 1 ml transferred to a glass vial. Amine-bearing mannosides (0.04 mmol) and EDC (34 mg, 0.22 mmol) were then added and the mixture stirred vigorously for 18 h at room temperature. The glycoconjugate solution was then dialysed against water for 16 h and the glycosylated probes collected and freeze dried.

Soluble particles suitable for coupling include fluorescent carbon dots and latex microbeads. Such particles are known in the art.

For example, the fluorescent carbon dots used in this study were prepared in accordance with the procedures set out in T. A. Swift, M. Duchi, S. A. Hill, D. Benito-Alifonso, R. L. Harniman, S. Sheikhac, S. A. Davis, A. M. Seddon, H. M. Whitney, M. C. Galan and T. A. A. Oliver, “Surface nanoparticle functionalization affects the physical and electronic structure of fluorescent carbon dots” Nanoscale, 2018, 10, 13908. An example synthesis of FCDs is shown in FIG. 2.

NMR of Mannose Functionalised FCD

Compound 7b wherein the particle is a CD.

1 H NMR (400 MHz, D₂O) δ 5.98-5.82 (m, CD), 4.88 (m, H-1), 4.27-4.12 (m, CD), 3.98-3.94 (m, H-2, CD), 3.94-3.87 (m, H-6a), 3.87-3.61 (m, H-3, H, 4, H-5, H-6b, OCHH, CD), 3.61-3.48 (m, OCHH), 3.48-3.29 (m, CD), 3.29-3.16 (m, CH₂NH), 3.16-3.02 (m, CD), 2.94-2.73 (m, CD), 1.71-1.43 (m, CH₂).

13C NMR (100 MHz, Key signals from HSQC, D₂O) δ 99.8 (C-1), 73.0 (C-5), 70.8 (C-3), 70.6 (CD), 70.1 (C-2), 67.9 (OCH₂), 66.9 (C-4), 61.0 (C-6), 54.3 (CD), 44.9 (CD), 42.6 (CH₂NH), 41.7 (CD), 39.1 (several signals, CD), 29.1 (CH₂), 28.4 (CH₂), 28.0 (CH₂), 25.9 (CH₂), 25.2 (CH₂).

Microbeads

The latex microbeads used in this study are Molecular Probes™ CML Latex Beads, 4% w/v, 10 μm commercially available from Fisher Scientific. These carboxylate modified (CML) latex particles are produced by copolymerizing carboxylic acid containing polymers. The result is a latex polymer particle with a highly charged, relatively hydrophilic and somewhat ‘fluffy’ surface layer. The CML particles are electrosterically stabilized, and are therefore safe in concentrations of electrolyte up to 1 M univalent salt. CML modified latex particles are negatively charged with a surface which has a polyelectrolyte character. It is only when the pH is ˜10 that all the carboxyl groups are ionized.

### Example 4—Agglutination Assay Methods of Mannoside Probes

Agglutination Assay Method A

Microsphere Probes Cause Agglutination

Agglutination is the clumping of microspheres to look like curdled milk. Agglutination is usually based on the very specific interaction between antigen with antibody. These microspheres have a much larger surface area than the bacteria which mimics conditions in the host body during infection. The larger surface area coupled with shear force favours adhesion of the bacteria to the probe. When a bacteria cell attaches to two or more beads, the beads clump together, hence the term agglutination. Agglutination tests have been around since 1956; these could be microspheres or latex agglutination tests (LAT). LAT have been applied to chemical analyte, bacterial, and fungal detection.

As the microsphere probes described herein specifically target the bacteria fimbriae, agglutination was chosen because it should only occur when the bacteria fimbriae are attached to the microsphere probe. Another reason is that the clumps are visible, and the unbound bacteria or probes do not need to be removed.

Bacteria Sample Preparation
1. Set up an overnight culture of E. Coli in TSB on a shaker incubator (37 C @ 200 rpm).
2. Dilute the overnight culture of E. Coli to a concentration of 10\hat{ }7 cfu/ml in TSB.

The assay protocol is as follows:

Sample Preparation
1. Pipette 20 ul E. Coli sample into Eppendorf.
2. Then add 10 ul of probe and mix solution for even distribution of beads. This could be done by vortex or by hand.
3. Vortex mix the probe and bacteria sample briefly.

Incubation at Room Temperature (22 C)
4. Vortex mix for 15 mins (1500 rpm with our LP vortex mixer in continuous mode).
5. Rest for 10 mins.

Data Collection
6. Pipette sample into standard microscope slide
7. Take 3 images at different locations on the spot at 10× magnification

Image Processing

Matlab was used to process the images collected. The single free-floating beads are identified separately to the clusters. The single beads are circular: width and length approximately the same and diameter of 20 μm. A cluster is defined as a group of beads close together which form a non-circular feature with area greater than 314.4 micron square.

Below are the processing steps
Take images and reconstruct circles around features.
Sum the number of clusters and cluster area in the 3 images of the blank sample respectively.
Similarly sum the number of clusters and cluster area in the 3 images of the sample with bacteria, respectively.

The sample with bacteria should have larger and more clusters than the bank.

Data Interpretation

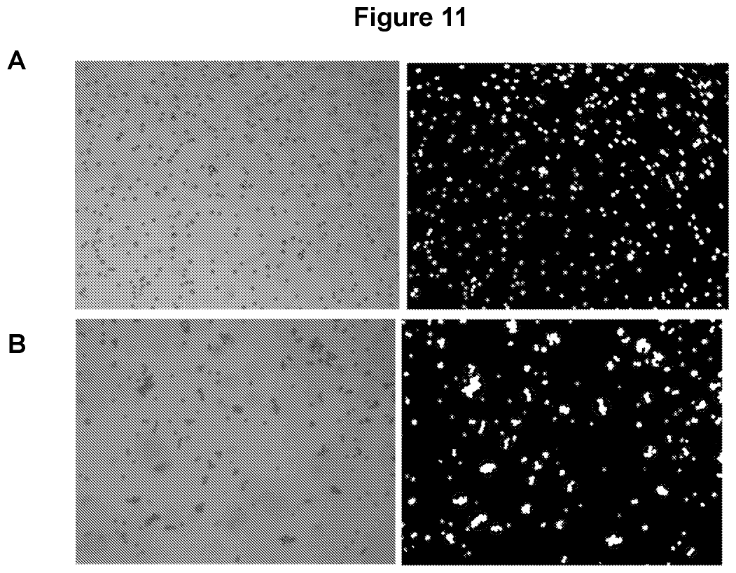

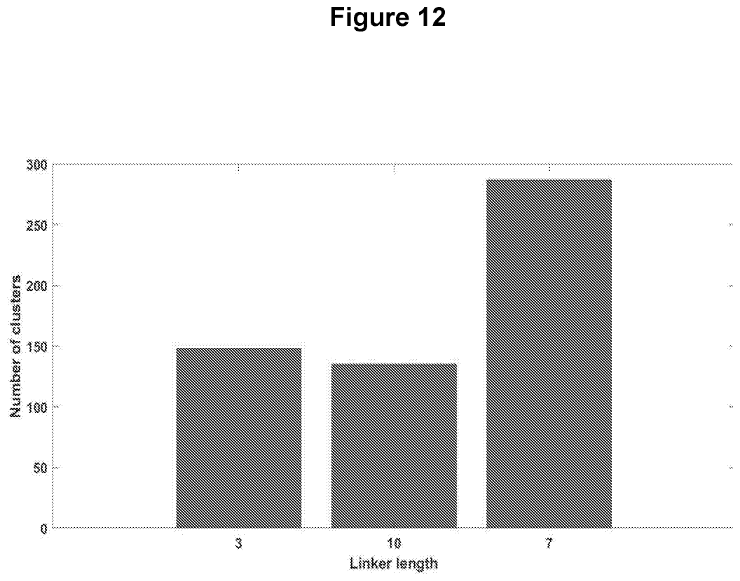

A bacteria positive sample is one where the ratio of free beads to clusters has shifted to minimal free beads and more and larger clusters. This can be seen, for instance, in FIG. 11. FIG. 11 shows microscope photographs of test samples mixed with a microsphere probe according to the invention. The test sample in FIG. 12A is without bacteria and FIG. 11B shows a test sample with bacteria having type 1 fimbriae. In both FIGS. 11A and 11B the left image is the original bright-field image and the right image is after processing. After image processing the single beads and clusters can be highlighted in different colours for convenience. For example, single beads can be circled in red and clusters are circled in blue. Clusters identified in the blanks are generally due to beads settling by each other, and the software reconstructs the feature as a cluster. From FIGS. 11A and 11B, more clusters are identified in the sample with bacteria, as can be seen in both the bright-field and processed images. There are a greater number of clusters and the clusters are larger.

Probe Screening Using Agglutination Assay Method A

The images in FIG. 11 were obtained according to the following procedure. The probes used in this assay correspond with compounds 7a-c, wherein the particle is a latex microbead having a diameter of 10 um. The microbead surface comprises carboxylic acid moieties and the remainder of the probe is installed using a peptidic coupling using EDC (1-ethyl-3-(3-dimethylaminopropyl)carbodiimide hydrochloride) as a coupling agent. FIG. 3A shows the result of a blank which contained no bacteria. FIG. 3B shows the result of the probe mixed with a sample containing BW25113 E. coli having type 1 fimbriae.

In a further experiment, the length of the glycosidic alkyl chain in was plotted against the number of clusters the probe was able to form, and the results can be seen in FIG. 12. It can clearly be seen that the C₇ glycosidic alkyl chain was optimal.

Agglutination Assay Method B—Nanosphere Probes Inhibit Agglutination

The agglutination assay is based on the principle that type-1 piliated bacteria such as FimH-producing Escherichia coli cause aggregation of mannan-containing Saccharomyces cerevisiae by lectin-specific interactions [Ofek, I., Mirelman, D. & Sharon, N. Adherence of Escherichia coli to human mucosal cells mediated by mannose receptors. Nature 265, 623-625 (1977)]. When FimH-specific sugars such as α-D-mannose and methyl α-_{D}-mannopyranoside are introduced, they prevent the interaction between the two microorganisms and disrupt agglutination. There are many variations to this assay, such as the use of guinea pig erythrocytes instead of yeast cells, but the present study has adapted the original technique with minor modifications [Hultgren, S. J., Schwan, W. R., Schaeffer, A. J. & Duncan, J. L., “Regulation of production of type 1 pili among urinary tract isolates of Escherichia coli” Infect. Immun. 54, 613-620 (1986); 3.

Mirelman, D., Altmann, G. & Eshdat, Y. “Screening of bacterial isolates for mannose-specific lectin activity by agglutination of yeasts”, J. Clin. Microbiol. 11, 328-331 (1980)].

To summarise, E. coli BW25113 and its Fim pilin-deficient mutant ΔfimA (from the Keio collection—Baba, T. et al. Construction of Escherichia coli K-12 in-frame, single-gene knockout mutants: the Keio collection. Mol. Syst. Biol. 2, 2006.0008 (2006)) were grown statically in LB broth for 24-40 h at 37° C. S. cerevisiae BY4741 was grown in YPD broth for 2-3 days at 30° C. with shaking. All cultures were adjusted to an OD₆₀₀ of 2.0 before centrifuged at 6,000×g for 1 min at room temperature. The pellets were washed once with PBS and re-suspended in the same buffer. 125 μl of S. cerevisiae suspension was spotted on a sterile surface, followed by a 50 μl overlay of either E. coli suspension. The spot was left at room temperature for 10-20 min with gentle agitation at 10 min to encourage agglutination. When testing fluorescent carbon dots for FimH-specific interactions, the nanomaterial was added to the E. coli suspension to a final concentration of 200 μg/ml, gently mixed and left to sit at room temperature for 1 min before being spotted.

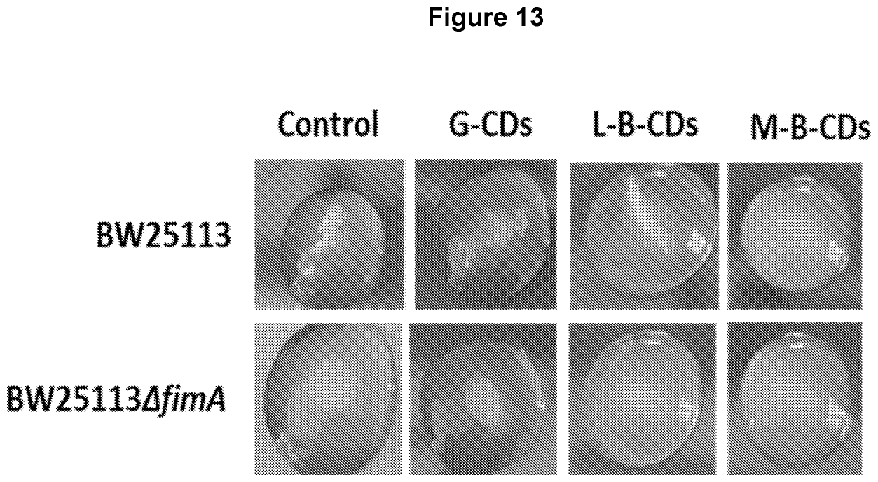

FIG. 13 is a summary of the results obtained for unfunctionalised green and lactosylated and mannosylated CDs. Control denotes no CDs added; G-CDs, unfunctionalised CDs; L-B-CDs, lactosylated blue CDs; M-B-CDs, mannosylated CDs. The mannosylated CD used in this study is compound 7b wherein the particle is a CD (i.e. the CD is functionalised with mannose via the C₇ alkyl glycosidic chain). The lactosylated CD is the lactose equivalent of the mannosylated CD. Agglutination was observed in all E. coli BW25113 spots except with the addition of mannosylated CDs. Inhibition of agglutination confirms that the mannosylated CDs are having FimH-specific interactions with the E. coli. All spots with Fim pilin-deficient E. coli BW25113ΔfimA showed no signs of agglutination, as expected. This assay confirms the FimH-specific interactions of mannosylated CDs.

| | Control | G-CDs | L-B-CDs | M-B-CDS |
|---|---|---|---|---|
| BW25113 | Full | Full | Full | No |
| | agglutination | agglutination | agglutination | agglutination |
| BW25113ΔfimA | No agglutination | No agglutination | No agglutination | No agglutination |

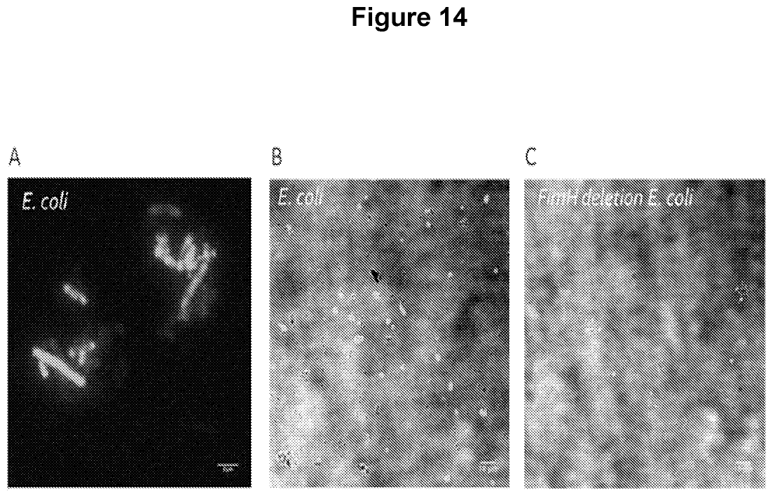

FIG. 14 shows the successful labelling of E. coli by mannose functionalised green quantum dot nanoparticles 3. The quantum dots were prepared according to established procedures (i.e. the procedure set out in ACS Omega 2018, 3, 8, 9822-9826). FIG. 6 shows confocal images of mannose-linker QD incubated with the BW25113 E. coli and the FimH knockout for 1 hour before fixation. A. Fluorescence channel showing labelling of the E. coli. B-C. Overlay of fluorescence and bright field channels of E. coli (B) and fimH knockout E. coli.

Many modifications may be made to the above examples without departing from the scope of the present invention as defined in the accompanying claims

1. A composition for the detection of target cellular material, the composition comprising:
a chemically modified substrate;
at least one binding moiety, wherein the binding moiety comprises a glycan; and
at least one linker covalently linked to the substrate and the binding moiety by a first and second bond.

2. The composition of claim 1, wherein the first bond is a peptide bond.

3. The composition of claim 1 wherein the second bond is a peptide bond, a glycosidic bond, or a bond formed by a 1,3-Huisgen-cycloaddition.

4. The composition of claim 1 wherein the substrate is a bead or a particle.

5. The composition of claim 4 wherein the substrate is selected from the group consisting of: microbeads, latex beads, magnetic beads, carbon dot, and gold nanoparticles.

6. The composition of claim 1, wherein the substrate is carboxyl acid or amine functionalised.

7. The composition of claim 1, wherein the glycan is selected from the group consisting of: Xylose; GalNAc, Gal-α-(1-4)-Gal; Gal; Man; Glc; GlcNAc; GalNAc-β-(1-4)-Gal; β-Gal; Gal-β-(1-4)-GlcNAc; GlcNAc-β-(1-3)-Gal-β-(1-4)-Glc; Glc; Gal-β-(1-3)-GalNAc; Gal-α-(1-3)-Gal; Gal-α-(1-6)-Glc; GlcNAc-β-(1-6)-GlcNAc; Fuc; GalNAc; Lac; Sorb and combinations thereof.

8. The composition of claim 1, wherein the chemically modified substrate comprises a plurality of glycans and linkers.

9. The composition of claim 7, wherein the glycan is a glycosylamine or a sugar acid.

10. The composition of claim 1, wherein the linker is attached to the substrate by peptidic coupling or triazole formation through a 1,3-Huisgen cycloaddition.

11. The composition of claim 1, wherein the linker is selected from the group consisting of a bifunctional amine; a carboxylic acid; an alkanolamine; an alkanolamide; and an aminocarboxylic acid.

12. The composition of claim 1, wherein the linker is selected from the group consisting of: 2-azidoethanol; 3-azidopropanol; 5-azidopentanol; 7-azidoheptanol; 10-azidodecanol; 2-aminoethanol; 3-aminopropanol; 5-aminopentanol; 7-aminoheptanol; 10-aminodecanol; 4,7,10-trioxa-1,13-diaminotridecane and carboxylated variants thereof.

13. The composition of claim 1, wherein the composition comprises a spacer.

14. The composition of claim 13, wherein the spacer is selected from the group consisting of: 2-(2-(2-(2-aminoethoxy)ethoxy)ethoxy)ethanol; and 4,7,10-trioxa-1,13-diaminotridecane.

15. The composition of claim 1, wherein the target cellular material is located on the cell surface of a microorganism.

16. The composition of claim 15, wherein the microorganism is selected from the group consisting of: Escherichia coli; Klebsiella pneumoniae; Proteus mirabilis; Enterobacter cloacae; Proteus vulgaris, Klebsiella aerogenes; Citrobacter freundii; Citrobacter kosieri; Staphylococcus saprophyticus; Staphylococcus aureus; Staphylococcus epidermidis; Enterococcus faecalis; Streptococcus agalactiae; Streptococcus pyogenes; Candida albicans; Neisseria gonorrhoeae and Treponema.

17. A method for the detection of target cellular material, the method comprising:
(i) providing a sample comprising cellular material;
(ii) adding to the sample the composition as described in claim 1; and;
(iii) measuring the level of agglutination in order to determine the presence of target cellular material in the sample.

18.-42. (canceled)

43. A kit for the detection of cellular material comprising:
(i) a composition comprising a chemically modified substrate, a binding moiety comprising a glycan, and a linker covalently linked to the substrate and the binding moiety as described in claim 1;
(ii) a buffer solution; and
(iii) a solvent.

44. (canceled)

45. The composition of claim 1 wherein the linker is one of the following structures: 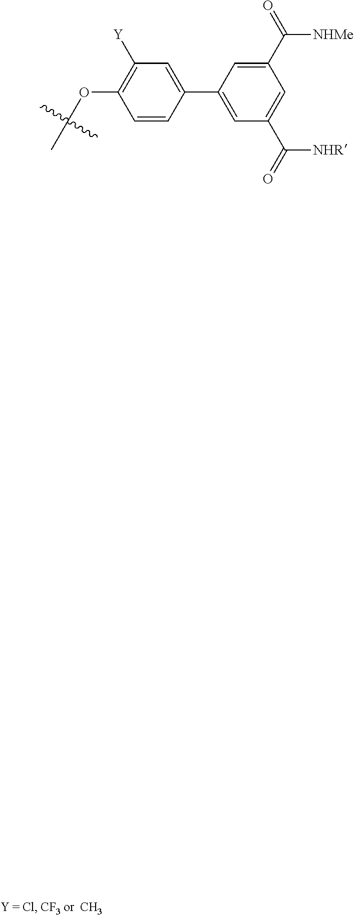
or combinations thereof.

46. The composition of claim 8, wherein the plurality of glycans are identical.
